(19) United States
(12) Patent Application Publication
(10) Pub. No.: US 2019/0079100 A1
(43) Pub. Date: Mar. 14, 2019

(54) METHODS OF GLYCOPROTEIN ANALYSIS

(71) Applicant: Momenta Pharmaceuticals, Inc., Cambridge, MA (US)

(72) Inventors: Desiree Tsao, Belmont, MA (US); James Anderson, Hudson, MA (US)

(73) Assignees: Momenta Pharmaceuticals, Inc., Cambridge, MA (US); Momenta Pharmaceuticals, Inc., Cambridge, MA (US)

(21) Appl. No.: 15/571,683

(22) PCT Filed: May 6, 2016
(86) PCT No.: PCT/US16/31298

§ 371 Date: Nov. 3, 2017

Related U.S. Application Data
(60) Provisional application No. 62/157,922, filed on May 6, 2015.

(51) Int. Cl. G01N 33/68; G01N 24/08; G01R 33/465

(52) U.S. Cl. CPC G01N 33/6854; G01N 2440/38; G01R 33/465; G01N 24/088

(57) ABSTRACT

Methods of assessing biosimilarity of proteins, e.g., therapeutic antibodies, are described.

### CROSS REFERENCE TO RELATED APPLICATIONS

This application claims the benefit of U.S. Provisional Application No. 62/157,922, filed May 6, 2015, the contents of which are hereby incorporated herein in their entirety.

### BACKGROUND

Therapeutic polypeptides are an important class of therapeutic biotechnology products, and therapeutic antibodies (including murine, chimeric, humanized and human antibodies and fragments thereof) account for the majority of therapeutic biologic products.

### SUMMARY OF THE INVENTION

The present disclosure provides, in part, methods for evaluating, identifying, analyzing and/or producing (e.g., manufacturing) a protein, e.g., a glycoprotein, e.g., an antibody and/or a biosimilar antibody, wherein a biosimilar antibody is an antibody approved for use in humans by a secondary approval process. In some instances, methods herein allow highly resolved evaluation of a protein (e.g., a glycoprotein, e.g., an antibody) useful for, inter alia, manufacturing and/or evaluating a protein such as a biosimilar antibody.

In certain aspects, the disclosure provides methods of manufacturing. Such methods can include providing (e.g., producing or expressing (e.g., in small scale or large scale cell culture) or manufacturing) or obtaining (e.g., receiving and/or purchasing from a third party (including a contractually related third party or a non-contractually-related (e.g., an independent) third party) a test protein (e.g., a test protein drug substance, e.g., a batch of a test protein drug substance), e.g., wherein the test protein (e.g., test protein drug substance, e.g., batch of a test protein drug substance) is not approved under a BLA; exposing a sample of the test protein (e.g., intact test protein, e.g., intact test protein drug substance) in a first state to a plurality of stressors to obtain a plurality of test protein (e.g., intact test protein, e.g., intact test protein drug substance) in a second state; acquiring (e.g., detecting, measuring, determining, receiving, or obtaining) a signal associated with higher-order structure of the test protein (e.g., intact test protein, e.g., intact test protein drug substance) in the first state and for each of the plurality of test protein (e.g., intact test protein, e.g., intact test protein drug substance) in the second state; acquiring (e.g., detecting, measuring, determining, receiving, or obtaining) a test protein delta between the signal associated with higher-order structure of the test protein drug substance in the first state and the signal associated with higher-order for each of the plurality of test protein drug substance in the second state; comparing the determined test protein deltas to corresponding target protein deltas of a target protein (e.g., target protein drug substance) to determine if the test protein deltas and the target protein deltas are tolerable; and processing the test protein (e.g., test protein drug substance, e.g., batch of test protein drug substance) as test protein drug product (e.g., for administration to a subject) if the test protein deltas and the target protein deltas are tolerable; or taking an alternative action if the test protein deltas and the target protein deltas are not tolerable. In some embodiments, the target protein has an amino acid sequence with at least 85% identity (e.g., 90, 95, 98, 99, or 100%) identity to the test protein. In some embodiments, the target protein is approved under a BLA. In some embodiments, the method further comprises producing a representation of the comparison of the test protein deltas and the target protein deltas.

In some embodiments, the first state of a test protein is a higher-order structure of the test protein in a first condition or set of conditions (e.g., first storage condition(s) and/or first condition(s) for obtaining a signal, e.g., first NMR conditions), and a second state of a test protein is a higher-order structure of the test protein in a second set of conditions (e.g., exposure to a stressor). In some embodiments, the first state is a native state (e.g., a state of a protein in standard, conventional, and/or customary storage conditions for the protein, or in standard, conventional, and/or customary conditions for acquiring a signal, e.g., an NMR signal). In some embodiments, the first state is a native state and the second state is a non-native state (e.g., a state of a protein in non-standard, non-conventional, and/or non-customary storage conditions for the protein, or in non-standard, non-conventional, and/or non-customary conditions for acquiring a signal, e.g., an NMR signal).

In some embodiments, one or more of the plurality of stressors comprises a condition that alters a higher-order structure of a protein and/or comprises an NMR shift agent. In some embodiments, one or more of the plurality of stressors include: increased or reduced time (e.g., a defined duration of minutes, hours, days, weeks, months, or years), elevated or reduced temperature (e.g., of about 5, 10, 15, 20, 25, 30, 35, 40, 45, 50, 55, 60, 65, 70, 75, or 80° C.), presence or absence of an oxidating agent, presence or absence of an acid or base, presence or absence of light (e.g., a defined level of light), presence or absence of an NMR shift reagent, all relative to a first set of conditions.

In some embodiments, detecting a signal comprises an NMR method. In some embodiments, the NMR method is one-dimensional NMR (1D-NMR), two-dimensional NMR (2D-NMR), correlation spectroscopy magnetic-angle spinning NMR (COSY-NMR), total correlated spectroscopy NMR (TOCSY-NMR), heteronuclear single-quantum coherence NMR (HSQC-NMR), heteronuclear multiple quantum coherence (HMQC-NMR), rotational nuclear overhauser effect spectroscopy NMR (ROESY-NMR), nuclear overhauser effect spectroscopy (NOESY-NMR), or a combination thereof.

In some embodiments, detecting a signal comprises an NMR method, and the signal associated with higher-order structure comprises one or more peaks of an NMR spectrum, e.g., 1, 2, 3, 4, 5, 6, 7, 8, 9, 10, 11, 12, 13, 14, 15, 16, 17, 18, 19, 20, 21, 22, 23, 24, 25, or more, peaks.

In some embodiments, detecting a signal comprises an NMR method, and the signal associated with higher-order structure comprises one or more points (e.g. point intensities) of an NMR spectrum, e.g., 100-100,000 points, 1,000-50,000 points, 500-5,000 points, 1,000-10,000 points, etc.

In some embodiments, the step of comparing comprises a statistical analysis (e.g., linear regression analysis) and the representation is a graphical representation, e.g., linear regression plot. In some embodiments, the representation is tolerable if it meets a predetermined value. In some embodiments, the predetermined value is an R² value of at least 0.8, 0.85, 0.9, 0.91, 0.92, 0.93, 0.94, 0.95, 0.96, 0.97, 0.98, 0.99, or 1. In some embodiments, corresponding target protein deltas are a historical record of the target protein.

In some embodiments, the test protein (e.g., test protein drug substance) and the target protein (e.g., target protein drug product) are glycoproteins. In some embodiments, the test protein and the target protein are antibodies. In some embodiments, the test protein and the target proteins are intact proteins. In some embodiments, the test protein and the target protein are antibody fragments, e.g., Fab fragments and/or Fc fragments.

In some instances, the processing step includes combining the test protein with an excipient or buffer. In some embodiments, the processing step includes, but is not limited to, one or more of: formulating the test protein; processing the test protein into a drug product; combining the test protein with a second component, e.g., an excipient or buffer; changing the concentration of the test protein in a preparation; lyophilizing the test protein; combining a first and second aliquot of the test protein to provide a third, larger, aliquot; dividing the test protein into smaller aliquots; disposing the test protein into a container, e.g., a gas or liquid tight container; packaging the test protein; associating a container comprising the test protein with a label (e.g., labeling); shipping or moving the test protein to a different location.

In some embodiments, the alternative action comprises one or more of disposing of the test protein (e.g., test protein drug substance, e.g., batch of test protein drug substance), classifying for disposal the test protein (e.g., test protein drug substance, e.g., batch of test protein drug substance), labeling the test protein (e.g., test protein drug substance, e.g., batch of test protein drug substance) for disposal, and reprocessing the test protein (e.g., test protein drug substance, e.g., batch of test protein drug substance).

In some instances, methods can further include, e.g., one or more of: memorializing the representation using a recordable medium (e.g., on paper or in a computer readable medium, e.g., in a Certificate of Testing, Material Safety Data Sheet (MSDS), batch record, or Certificate of Analysis (CofA)); informing a party or entity (e.g., a contractual or manufacturing partner, a care giver or other end-user, a regulatory entity, e.g., the FDA or other U.S., European, Japanese, Chinese or other governmental agency, or another entity, e.g., a compendial entity (e.g., U.S. Pharmacopoeia (USP)) or insurance company) of the representation.

In another aspect, the disclosure provides methods of manufacturing. Such methods can include providing (e.g., producing or expressing (e.g., in small scale or large scale cell culture) or manufacturing) or obtaining (e.g., receiving and/or purchasing from a third party (including a contractually related third party or a non-contractually-related (e.g., an independent) third party) a test protein (e.g., a test protein drug substance, e.g., a batch of a test protein drug substance), e.g., wherein the test protein (e.g., test protein drug substance, e.g., batch of test protein drug substance) is not approved under a BLA; exposing a sample of the test protein (e.g., intact test protein, e.g., intact test protein drug substance, e.g., batch of intact test protein drug substance) in a first state to a stressor to obtain a test protein (e.g., intact test protein, e.g., intact test protein drug substance) in a second state; acquiring (e.g., detecting, measuring, determining, receiving, or obtaining) a signal associated with higher-order structure of the test protein (e.g., intact test protein, e.g., intact test protein drug substance) in the first state and for the test protein (e.g., intact test protein, e.g., intact test protein drug substance) in the second state; acquiring (e.g., detecting, measuring, determining, receiving, or obtaining) a test protein delta between the signal associated with higher-order structure of the test protein drug substance in the first state and the signal associated with higher-order for the test protein drug substance in the second state; comparing the determined test protein delta to a corresponding target protein delta of a target protein (e.g., target protein drug substance) to determine if the test protein delta and the target protein delta are tolerable; and processing the test protein (e.g., test protein drug substance, e.g., batch of test protein drug substance) as test protein drug product (e.g., for administration to a subject) if the test protein delta and the target protein delta are tolerable; or taking an alternative action if the test protein delta and the target protein delta are not tolerable. In some embodiments, the target protein has an amino acid sequence with at least 85% identity (e.g., 90, 95, 98, 99 or 100% identity) to the test protein. In some embodiments, the target protein is approved under a BLA. In some embodiments, the method further comprises producing a representation of the comparison of the test protein delta and the target protein delta.

In some embodiments, the first state of a test protein is a higher-order structure of the test protein in a first condition or set of conditions (e.g., first storage condition(s) and/or first condition(s) for obtaining a signal, e.g., first NMR conditions), and a second state of a test protein is a higher-order structure of the test protein in a second set of conditions (e.g., exposure to a stressor). In some embodiments, the first state is a native state (e.g., a state of a protein in standard, conventional, and/or customary storage conditions for the protein, or in standard, conventional, and/or customary conditions for acquiring a signal, e.g., an NMR signal). In some embodiments, the first state is a native state and the second state is a non-native state (e.g., a state of a protein in non-standard, non-conventional, and/or non-customary storage conditions for the protein, or in non-standard, non-conventional, and/or non-customary conditions for acquiring a signal, e.g., an NMR signal).

In some embodiments, the stressor comprises a condition that alters a higher-order structure of a protein and/or comprises an NMR shift agent. In some embodiments, the stressor includes: increased or reduced time (e.g., a defined duration of minutes, hours, days, weeks, months, or years), elevated or reduced temperature (e.g., of about 5, 10, 15, 20, 25, 30, 35, 40, 45, 50, 55, 60, 65, 70, 75, or 80° C.), presence or absence of an oxidating agent, presence or absence of an acid or base, presence or absence of light (e.g., a defined level of light), presence or absence of an NMR shift reagent, all relative to a first set of conditions.

In some embodiments, detecting a signal comprises an NMR method. In some embodiments, the NMR method is one-dimensional NMR (1D-NMR), two-dimensional NMR (2D-NMR), correlation spectroscopy magnetic-angle spinning NMR (COSY-NMR), total correlated spectroscopy NMR (TOCSY-NMR), heteronuclear single-quantum coherence NMR (HSQC-NMR), heteronuclear multiple quantum coherence (HMQC-NMR), rotational nuclear overhauser effect spectroscopy NMR (ROESY-NMR), nuclear overhauser effect spectroscopy (NOESY-NMR), or a combination thereof.

In some embodiments, detecting a signal comprises an NMR method, and the signal associated with higher-order structure comprises one or more peaks of an NMR spectrum, e.g., 1, 2, 3, 4, 5, 6, 7, 8, 9, 10, 11, 12, 13, 14, 15, 16, 17, 18, 19, 20, 21, 22, 23, 24, 25, or more, peaks.

In some embodiments, detecting a signal comprises an NMR method, and the signal associated with higher-order structure comprises one or more points (e.g. point intensities) of an NMR spectrum, e.g., 100-100,000 points, 1,000-50,000 points, 500-5,000 points, 1,000-10,000 points, etc.

In some embodiments, the step of comparing comprises a statistical analysis (e.g., linear regression analysis) and the representation is a graphical representation, e.g., linear regression plot. In some embodiments, the representation is tolerable if it meets a predetermined value. In some embodiments, the predetermined value is an R² value of at least 0.8, 0.85, 0.9, 0.91, 0.92, 0.93, 0.94, 0.95, 0.96, 0.97, 0.98, 0.99, or 1. In some embodiments, a corresponding target protein delta is a historical record of the target protein.

In some embodiments, the test protein (e.g., test protein drug product) and the target protein (e.g., target protein drug product) are glycoproteins. In some embodiments, the test protein and the target protein are antibodies. In some embodiments, the test protein and the target proteins are intact proteins. In some embodiments, the test protein and the target protein are antibody fragments, e.g., Fab fragments and/or Fc fragments.

In some instances, the processing step includes combining the test protein with an excipient or buffer. In some embodiments, the processing step includes, but is not limited to, one or more of: formulating the test protein; processing the test protein into a drug product; combining the test protein with a second component, e.g., an excipient or buffer; changing the concentration of the test protein in a preparation; lyophilizing the test protein; combining a first and second aliquot of the test protein to provide a third, larger, aliquot; dividing the test protein into smaller aliquots; disposing the test protein into a container, e.g., a gas or liquid tight container; packaging the test protein; associating a container comprising the test protein with a label (e.g., labeling); shipping or moving the test protein to a different location.

In some embodiments, the alternative action comprises one or more of disposing of the test protein (e.g., test protein drug substance, e.g., batch of test protein drug substance), classifying for disposal the test protein (e.g., test protein drug substance, e.g., batch of test protein drug substance), labeling the test protein (e.g., test protein drug substance, e.g., batch of test protein drug substance) for disposal, and reprocessing the test protein (e.g., test protein drug substance, e.g., batch of test protein drug substance).

In some instances, methods can further include, e.g., one or more of: memorializing the representation using a recordable medium (e.g., on paper or in a computer readable medium, e.g., in a Certificate of Testing, Material Safety Data Sheet (MSDS), batch record, or Certificate of Analysis (CofA)); informing a party or entity (e.g., a contractual or manufacturing partner, a care giver or other end-user, a regulatory entity, e.g., the FDA or other U.S., European, Japanese, Chinese or other governmental agency, or another entity, e.g., a compendial entity (e.g., U.S. Pharmacopoeia (USP)) or insurance company) of the representation.

In some aspects, the disclosure provides methods of manufacture, e.g., manufacturing a drug product or drug substance. Such methods can include providing (e.g., producing or expressing (e.g., in small scale or large scale cell culture) or manufacturing) or obtaining (e.g., receiving and/or purchasing from a third party (including a contractually related third party or a non-contractually-related (e.g., an independent) third party) a first preparation of a test glycoprotein drug substance; acquiring (e.g., detecting, measuring, determining, quantitating, receiving, or obtaining) a first 2D NMR signal profile of the first preparation; providing (e.g., producing or expressing (e.g., in small scale or large scale cell culture) or manufacturing) or obtaining (e.g., receiving and/or purchasing from a third party (including a contractually related third party or a non-contractually-related (e.g., an independent) third party) a second preparation of a target glycoprotein drug product, wherein the target glycoprotein has an amino acid sequence with at least 85% identity (e.g., 90, 95, 98, or 100% identity) to the test glycoprotein; acquiring (e.g., detecting, measuring, determining, quantitating, receiving, or obtaining) a second 2D NMR signal profile of the second preparation; comparing the first 2D NMR signal profile to the second 2D NMR profile to produce a representation; and processing the preparation of the test glycoprotein drug substance as drug product if the representation is tolerable; or disposing, marking for disposal, authorizing disposal and/or directing disposal of the test glycoprotein if the representation is not tolerable.

In some embodiments, a 2D NMR signal profile is from a 2D NMR spectrum. In some embodiments, a 2D NMR spectrum is a correlation spectroscopy magnetic-angle spinning NMR (COSY-NMR) spectrum, total correlated spectroscopy NMR (TOCSY-NMR) spectrum, heteronuclear single-quantum coherence NMR (HSQC-NMR) spectrum, heteronuclear multiple quantum coherence (HMQC-NMR) spectrum, rotational nuclear overhauser effect spectroscopy NMR (ROESY-NMR) spectrum, nuclear overhauser effect spectroscopy (NOESY-NMR) spectrum, or a combination thereof. In some embodiments, the spectrum is a 2D ¹H-¹³C correlation spectrum, e.g., ¹H-¹³C HMQC spectrum.

In some embodiments, the step of comparing comprises a statistical analysis (e.g., linear regression analysis) and the representation is a linear regression plot. In some embodiments, the representation is tolerable if it meets a threshold or predetermined value. In some embodiments, a threshold or predetermined value is an R² value of at least 0.8, 0.85, 0.9, 0.91, 0.92, 0.93, 0.94, 0.95, 0.96, 0.97, 0.98, 0.99, or 1.

In some embodiments, the test glycoprotein and the target glycoprotein are antibodies. In some embodiments, the test glycoprotein and the target glycoprotein are intact glycoproteins, e.g., intact antibodies. In some embodiments, the test glycoprotein and the target glycoprotein are antibody fragments, e.g., Fab fragments and/or Fc fragments. In some embodiments, the first preparation comprises about 10 mg/mL to about 150 mg/mL of the test glycoprotein (e.g., about 20 to 140, about 30 to about 130, about 40 to about 120, about 50 to about 110, about 60 to about 100, about 70 to about 90, about 10, 20, 30, 40, 50, 60, 70, 80, 90, 100, 110, 120, 130, 140, or 150 mg/mL test glycoprotein). In some embodiments, the second preparation comprises about 10 mg/mL to about 150 mg/mL of the target glycoprotein (e.g., about 20 to 140, about 30 to about 130, about 40 to about 120, about 50 to about 110, about 60 to about 100, about 70 to about 90, about 10, 20, 30, 40, 50, 60, 70, 80, 90, 100, 110, 120, 130, 140, or 150 mg/mL target glycoprotein).

In some embodiments, the test glycoprotein and/or the target glycoprotein is approved under a biologics license application (BLA) under Section 351(a) of the Public Health Service (PHS) Act. In some embodiments, the test glycoprotein and/or the target glycoprotein is not approved under a BLA under Section 351(a) of the PHS Act. In some embodiments, the test glycoprotein is not approved under a BLA under Section 351(a) of the PHS Act, and the target glycoprotein is approved under a BLA under Section 351(a) of the PHS Act.

In some instances, methods can further include, e.g., one or more of: memorializing the representation using a recordable medium (e.g., on paper or in a computer readable medium, e.g., in a Certificate of Testing, Material Safety Data Sheet (MSDS), batch record, or Certificate of Analysis (CofA)); informing a party or entity (e.g., a contractual or manufacturing partner, a care giver or other end-user, a regulatory entity, e.g., the FDA or other U.S., European, Japanese, Chinese or other governmental agency, or another entity, e.g., a compendial entity (e.g., U.S. Pharmacopoeia (USP)) or insurance company) of the representation.

In some instances, the processing step includes combining the test glycoprotein with an excipient or buffer. In some embodiments, the processing step includes, but is not limited to, one or more of: formulating the test glycoprotein; processing the test glycoprotein into a drug product; combining the test glycoprotein with a second component, e.g., an excipient or buffer; changing the concentration of the test glycoprotein in a preparation; lyophilizing the test glycoprotein; combining a first and second aliquot of the test glycoprotein to provide a third, larger, aliquot; dividing the test glycoprotein into smaller aliquots; disposing the test glycoprotein into a container, e.g., a gas or liquid tight container; packaging the test glycoprotein; associating a container comprising the test glycoprotein with a label (e.g., labeling); shipping or moving the test glycoprotein to a different location.

In another aspect, the disclosure provides a method of comparing a test protein and a target protein. Such methods can include providing (e.g., producing or expressing (e.g., in small scale or large scale cell culture) or manufacturing) or obtaining (e.g., receiving and/or purchasing from a third party (including a contractually related third party or a non-contractually-related (e.g., an independent) third party) a test protein (e.g., a test protein drug substance, e.g., a batch of a test protein drug substance), e.g., wherein the test protein (e.g., test protein drug substance) is not approved under a BLA; exposing a sample of the test protein (e.g., test protein drug substance, e.g., batch of test protein drug substance) in a first state to a plurality of stressors to obtain a plurality of test protein (e.g., test protein drug product) in a second state; acquiring (e.g., detecting, measuring, determining, receiving, or obtaining) a signal associated with higher-order structure of the test protein (e.g., test protein drug product) in the first state and for each of the plurality of test protein (e.g., test protein drug product) in the second state; acquiring (e.g., detecting, measuring, determining, receiving, or obtaining) a test protein delta between the signal associated with higher-order structure of the test protein drug product in the first state and the signal associated with higher-order for each of the plurality of test protein drug product in the second state; comparing the determined test protein deltas to corresponding target protein deltas of a target protein (e.g., target protein drug product) to produce a representation, wherein the target protein has an amino acid sequence at least 98% identical to the test protein, and wherein the target protein is approved under a BLA; thereby comparing the test protein and the target protein.

### Definitions

As used herein, a “glycoprotein” refers to amino acid sequences that include one or more oligosaccharide chains (e.g., glycans) covalently attached thereto. Exemplary amino acid sequences include peptides, polypeptides and proteins. Exemplary glycoproteins include glycosylated antibodies and antibody-like molecules (e.g., Fc fusion proteins). Exemplary antibodies include monoclonal antibodies and/or fragments thereof, polyclonal antibodies and/or fragments thereof, and Fc domain containing fusion proteins (e.g., fusion proteins containing the Fc region of IgG1, or a glycosylated portion thereof).

As used herein, a “glycoprotein preparation” is a composition or mixture that includes at least one glycoprotein. In some instances, a glycoprotein preparation (e.g., such as a glycoprotein drug substance or a precursor thereof) can be a sample from a proposed or test batch of a drug substance or drug product.

As used herein, a “batch” of a glycoprotein preparation refers to a single manufacturing run of the glycoprotein. Evaluation of different batches thus means evaluation of different manufacturing runs or batches.

As used herein, “sample(s)” refer to separately procured samples. In some embodiments, evaluation of separate samples includes evaluation of different commercially available containers or vials of the same batch or from different batches.

As used herein, “acquire” or “acquiring” (e.g., “acquiring information”) means obtaining possession of a physical entity, or a value, e.g., a numerical value, by “directly acquiring” or “indirectly acquiring” the physical entity or value. “Directly acquiring” means performing a process (e.g., performing an assay or test on a sample) to obtain the physical entity or value. “Indirectly acquiring” refers to receiving the physical entity or value from another party or source (e.g., a third party laboratory that directly acquired the physical entity or value). “Directly acquiring” a physical entity includes performing a process, e.g., analyzing a sample, that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. “Directly acquiring” a value includes performing a process that includes a physical change in a sample or another substance, e.g., performing an analytical process (e.g., an NMR process) which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

As used herein, the term “approximately” or “about,” as applied to one or more values of interest, refers to a value that is similar to a stated reference value. In certain embodiments, the terms “approximately” or “about” refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less of the stated reference value.

In general, a “protein”, as used herein, is a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means.

The term “protein preparation” as used herein refers to a mixture of proteins obtained according to a particular production method. The proteins in a protein preparation may be the same or different, i.e., a protein preparation may include several copies of the same protein and/or a mixture of different proteins. The production method will generally include a recombinant preparation step using cultured cells that have been engineered to express the proteins in the protein preparation (or to express the proteins at a relevant level or under relevant conditions). The production method may further include an isolation step in which proteins are isolated from certain components of the engineered cells (e.g., by lysing the cells and pelleting the protein component by centrifugation). The production method may also include a purification step in which the proteins in the protein preparation are separated (e.g., by chromatography) from other cellular components, e.g., other proteins or organic components that were used in earlier steps. It will be appreciated that these steps are non-limiting and that any number of additional productions steps may be included. Different protein preparations may be prepared by the same production method but on different occasions (e.g., different batches). Alternatively, different protein preparations may be prepared by different production methods. Two production methods may differ in any way (e.g., expression vector, engineered cell type, culture conditions, isolation procedure, purification conditions, etc.).

As used herein, the terms “biologic”, “biotherapeutic”, and “biologic product” are used interchangeably to refer to peptide and protein products. For example, biologics herein include naturally derived or recombinant products expressed in cells, such as, e.g., proteins, glycoproteins, fusion proteins, growth factors, vaccines, blood factors, thrombolytic agents, hormones, interferons, interleukin based products, monospecific (e.g., monoclonal) antibodies, therapeutic enzymes. Some biologics are approved under a “Biologics License Application” or “BLA”, under section 351(a) of the Public Health Service (PHS) Act, whereas biosimilar and interchangeable biologics referencing a BLA as a reference product are licensed under section 351(k) of the PHS Act. Section 351 of the PHS Act is codified as 42 U.S.C. 262. Other biologics may be approved under section 505(b)(1) of the Federal Food and Cosmetic Act, or as abbreviated applications under sections 505(b)(2) and 505(j) of the Hatch Waxman Act, wherein section 505 is codified 21 U.S.C. 355.

As used herein, “approval” refers to a procedure by which a regulatory entity, e.g., the FDA or EMEA, approves a candidate for therapeutic or diagnostic use in humans or animals. As used herein, a “primary approval process” is an approval process which does not refer to a previously approved protein, e.g., it does not require that the protein being approved have structural or functional similarity to a previously approved protein, e.g., a previously approved protein having the same primary amino acid sequence or a primary amino acid sequence that differs by no more than 1, 2, 3, 4, 5, or 10 residues or that has 98% or more sequence identity. In embodiments the primary approval process is one in which the applicant does not rely, for approval, on data, e.g., clinical data, from a previously approved product. Exemplary primary approval processes include, in the U.S., a Biologics License Application (BLA), or supplemental Biologics License Application (sBLA), a New Drug Application (NDA) under 505(b)(1) of the Federal Food and Cosmetic Act, and in Europe an approval in accordance with the provisions of Article 8(3) of the European Directive 2001/83/EC, or an analogous proceeding in other countries or jurisdictions.

As used herein, a “secondary approval process” is an approval process that refers to clinical data for a previously approved product. In embodiments, a secondary approval requires that the product being approved have structural and/or functional similarity to a previously approved product, e.g., a previously approved protein having the same primary amino acid sequence or a primary amino acid sequence that differs by no more than 1, 2, 3, 4, 5, or 10 amino acid residues or that has at least 98%, 99% or more (100%) sequence identity. In embodiments a secondary approval process is one in which the applicant relies, for approval, on clinical data from a previously approved product. Exemplary secondary approval processes include, in the U.S., an approval under 351(k) of the Public Health Service Act or under section 505(j) or 505(b)(2) of the Hatch Waxman Act and in Europe, an application in accordance with the provisions of Article 10, e.g., Article 10(4), of the European Directive 2001/83/EC, or an analogous proceeding in other countries or jurisdictions.

As used herein, a “target protein” is any protein of interest to which comparison with a second or “test” protein is desired. An exemplary target protein is an antibody, e.g., a CDR-grafted, humanized or human antibody. Other target proteins include glycoproteins, cytokines, hematopoietic proteins, soluble receptor fragments, and growth factors. In some embodiments, a target protein is a commercially available, or approved, biologic that defines or provides the basis against which a test protein is measured or evaluated. In embodiments a target protein is commercially available for therapeutic use in humans or animals. In embodiments a target protein was approved for use in humans or animals by a primary approval process. In embodiments a target protein is a reference listed drug for a secondary approval process. Exemplary target proteins include those described herein.

A “signal”, as used herein, refers to a signal or representation obtained from NMR and associated with presence of one or more chemical compounds and/or structural characteristics. In some embodiments, a signal is a peak, or point therein, or cross-peak in an NMR spectrum.

A “signal integral”, as used herein, refers to magnitude of a particular signal. In some embodiments, a signal integral is obtained by measuring signal area and/or signal volume, e.g., in an NMR spectrum.

A “signal associated with higher-order structure”, as used herein, refers to a collection of one or more signals obtained for a protein wherein a signal is associated with an NMR peak with a signal to noise ratio of greater than 3, for example, greater than 4, 5, 6, 7, 8, 9, 10. In some embodiments, a signal associated with higher-order structure of a protein includes signals associated with about 1-40 (e.g., about 1-30, e.g., 1-20, e.g., 1-10) of representative peaks of an NMR spectrum.

As used herein, a “stressor” refers to any agent or condition that causes a detectable shift and/or change in an NMR response. For example, a stressor induces a shift of a protein from a first state to a second state. In some instances, a stressor can induce a conformational change of the protein, e.g., can induce a change from a first conformation to a second conformation. Exemplary stressors capable of inducing a conformational change include, without limitation, time (e.g., a defined duration of minutes, hours, days, weeks, months, etc.), temperature (e.g., elevated or reduced temperature), oxidating agents, acids or bases, or light. In some instances, a stressor can induce a change in NMR response. Exemplary stressors capable of inducing a change in NMR response include, without limitation, NMR shift reagents (e.g., one or more of deuterium or 4-hydroxy-2,2,6,6-tetramethyl-piperidine-1-oxyl (TEMPOL)).

As used herein, a “delta” is a quantitative or qualitative difference between a first state of a protein (e.g., before exposure to one or more stressors) and a second state of a protein (e.g., after exposure to one or more stressors). In some instances, a delta is a difference between a signal associated with higher-order structure of a protein before exposure to a stressor(s) and a signal associated with higher-order structure of a protein after exposure to a stressor(s). In some embodiments, a “test protein delta” includes one or more differences between one or more relative peak intensities of a test protein in a first state (e.g., before exposure to one or more stressor(s)) and one or more relative peak intensities of a test protein in a second state (e.g., after exposure to one or more stressor(s)). In some embodiments, a “target protein delta” includes one or more differences between one or more relative peak intensities of a target protein in a first state (e.g., before exposure to one or more stressor(s)) and one or more relative peak intensities of a target protein in a second state (e.g., after exposure to one or more stressor(s)).

As used herein, a “representation” is a numeric or graphical representation of a comparison of a test protein delta and a target protein delta. In some instances, a representation is produced using a statistical analysis method. In some instances, a representation is a linear regression plot.

“Tolerable”, as used herein, refers to an range of acceptability for a pair of compared deltas, e.g., for a test protein delta and a target protein delta. In some instances, a comparison herein is an assessment or measure of variability between a test protein delta and a target protein delta, and such compared deltas are tolerable if the variability between them does not exceed (e.g., as determined using a given statistical method) the variability of deltas determined for multiple distinct batches (e.g., 2, 3, 4, 5, or more batches) of such target protein, e.g., assessed using the same stressor(s) and same NMR. In some instances, a comparison is tolerable if it meets a predetermined value (e.g., obtained by assessing multiple batches of target protein, as described above). In some instances, comparison of deltas is performed using a representation. In some instances, a representation is a linear regression plot, and is tolerable if a determined R² value derived therefrom is greater than or equal to 0.9, 0.91, 0.92, 0.93, 0.94, 0.95, 0.96, 0.97, 0.98, or 0.99, or is equal to 1.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls. The section headings used herein are for organizational purposes only and are not to be construed as limiting the subject matter described in any way. The present application also incorporates by reference the entire contents of a U.S. Provisional Application filed on May 6, 2015 under Attorney Docket No. 2010403-0048 (M0130PRO).

These, and other aspects of the invention, are described in more detail below and in the claims.

### BRIEF DESCRIPTION OF THE DRAWINGS

FIG. 1 is a 2D HMQC spectrum showing methyl peaks for glycosylated target antibody. Peaks 10, 11 and 12 are from the glycan part of the target antibody.

FIG. 2 is an overlay of 2D HMQC spectra showing methyl peaks for the target antibody (blue) and the test antibody (red). The spectra have been shifted so that both sets of peaks are visible.

FIG. 3 is an overlay of 2D HMQC spectra showing methyl peaks for the target antibody (red) and non-target antibody 1 (blue). The spectra have been shifted so that both sets of peaks are visible.

FIG. 4 is an overlay of 2D HMQC spectra showing methyl peaks for the target antibody (red) and non-target antibody 2 (blue). The spectra have been shifted so that both sets of peaks are visible.

FIG. 5A is a linear regression plot of relative peak intensity of the target antibody versus non-target antibody 2 at 35 C. FIG. 5B is a linear regression plot of relative peak intensity of the target antibody versus non-target antibody 2 at 55° C. FIG. 5C is a linear regression plot of difference of relative peak intensities at 55° C. and 35° C. for the target antibody versus non-target antibody 2.

FIG. 6A is a linear regression plot of relative peak intensity of the target antibody versus test antibody 1 in the presence of a Tb⁺³ shift agent. FIG. 6B is a linear regression plot of differences in relative peak intensities of the target antibody versus test antibody 1 in the presence of the Tb⁺³ shift agent and the absence of the Tb⁺³ shift agent.

FIG. 7A is a linear regression plot of relative peak intensity of the target antibody versus test antibody 2 in the presence of TempoL. FIG. 7B is a linear regression plot of differences in relative peak intensities of the target antibody versus test antibody 2 in the presence of TempoL and the absence of TempoL. FIG. 7C is a linear regression plot of differences in relative peak intensities of the target antibody versus non-target antibody 2 in the presence of TempoL and the absence of TempoL.

FIG. 8A is a linear regression plot of relative peak intensity of the target antibody versus non-target antibody 1 at 80% D₂O at 55° C. FIG. 8B is a linear regression plot of differences in relative peak intensities of the target antibody versus non-target antibody 1 in the presence of 80% D₂O at 55° C. and the absence of 80% D₂O at 55° C.

FIG. 9A is a linear regression plot of point intensities from 6.49 ppm to 12.00 ppm of the target antibody versus test antibody 2 at 35° C. FIG. 9B is a linear regression plot of point intensities from 6.49 ppm to 12.00 ppm of the target antibody versus test antibody 2 at 55° C. FIG. 9C is a linear regression plot of point intensities from 6.49 ppm to 12.00 ppm of the target antibody versus non-target antibody 1 at 55° C.

### DETAILED DESCRIPTION

The present disclosure is based, in part, on the discovery that assessment by NMR of the behavior of a protein exposed to certain stressors can be used to predict biosimilarity, e.g., to manufacture biosimilar antibodies. For example, the present disclosure describes that NMR can be used to assess the behavior of a target protein exposed to a stressor and that such behavior can be compared to the behavior of a test protein exposed to the same stressor, and that biosimilarity can be determined if the two compared behaviors are tolerably comparable.

In some instances, methods described herein can be used to detect shifts of a test protein from a first to a second state following exposure to one or more stressors, which shifts of a test protein can be compared to corresponding shifts of a target protein in order to assess biosimilarity. Accordingly, the present disclosure provides strategies to assess biosimilarity of a protein (e.g., an antibody) to a target protein (e.g., a target antibody), e.g., during one or more stages of process development and/or production of a biosimilar product.

### Analysis Methods

Exposure of a protein to one or more stressors described herein can induce a shift in the protein from a first state to a second state, which can be assessed using methods such as NMR. In some instances, such a shift of a test protein from a first state to a second state can be compared to a corresponding shift of a target protein from a first state to a second state, e.g., to assess a level of similarity between the test and target proteins. Thus, in some embodiments, (i) a signal associated with higher-order structure of a first protein (e.g., a test protein) before exposure to one or more stressors is compared to a signal associated with higher-order structure of the first protein (e.g., the test protein) after exposure to one or more stressors, and a difference in the signals associated with higher-order structure of the first protein is assessed (i.e., a test protein delta is determined); (ii) a signal associated with higher-order structure of a second protein (e.g., a target protein) before exposure to one or more stressors is compared to a signal associated with higher-order structure of the second protein (e.g., the target protein) after exposure to one or more stressors, and a difference in the signals associated with higher-order structure of the second protein is assessed (i.e., a target protein delta is determined); and (iii) difference in signals associated with higher-order structure of the first protein (i.e., test protein delta) is compared to difference in signals associated with higher-order structure of the second protein (i.e., target protein delta), e.g., to assess level of similarity between the first and second proteins (e.g., between the test protein and the target protein).

In some instances, NMR is used to analyze signals associated with higher-order structure of a protein, as described herein.

### NMR Methods

In some embodiments, methods described herein utilize nuclear magnetic resonance (NMR) methods to detect signals, e.g., signals associated with higher-order structure of proteins (e.g., test proteins and/or target proteins described herein). Any known NMR method can be used to detect signal(s) utilized in methods of the disclosure. Exemplary nuclear magnetic resonance (NMR) include, but are not limited to, one-dimensional NMR (1D-NMR), two-dimensional NMR (2D-NMR), correlation spectroscopy magnetic-angle spinning NMR (COSY-NMR), total correlated spectroscopy NMR (TOCSY-NMR), heteronuclear single-quantum coherence NMR (HSQC-NMR), heteronuclear multiple quantum coherence (HMQC-NMR), rotational nuclear overhauser effect spectroscopy NMR (ROESY-NMR), nuclear overhauser effect spectroscopy (NOESY-NMR), and combinations thereof. A protein can be analyzed with, or without, a label (e.g., a label detectable by NMR).

Any known NMR equipment capable of detecting and/or measuring a signal associated with higher-order structure can be used in methods of the disclosure. For example, NMR spectrometers are commercially available at, e.g., Brucker Corp. and Thermo Scientific.

In some instances, a signal associated with higher-order structure of a protein is obtained by performing NMR on a protein (e.g., a sample of a protein preparation) to obtain an NMR spectrum comprising peaks (or points therein) or cross-peaks (“signals”). In some instances, a signal associated with higher-order structure of a protein includes one or more representative peaks (signals). In some instances, such representative peaks can be randomly chosen. In some embodiments, representative peaks can include one or more major or predominant peaks. In some embodiments, representative peaks include one or more amide peaks, one or more aromatic peaks, and/or one or more methyl peaks. For example, an NMR spectrum can be a 1D NMR spectrum, and a signal associated with higher-order structure includes one or more peaks from about 9 ppm to about −1.5 ppm. In some embodiments, a signal associated with higher-order structure of a protein includes 1, 2, 3, 4, 5, 10, 15, 20, 25, 30, 35, 40 or more peaks.

In some embodiments, representative peaks in an NMR spectrum are quantified. For example, magnitude of each representative peak can be obtained by measuring signal area and/or signal volume to yield a “signal integral” for a representative peak. In some instances, a representative peak is quantified as relative peak intensity.

In some embodiments, a signal associated with higher-order structure of a protein includes points associated with one or more representative peaks of an NMR spectrum. In some instances, a signal associated with higher-order structure of a protein includes point intensities over one or more regions of an NMR spectrum. For example, an NMR spectrum can be a 1D proton NMR spectrum, and a signal associated with higher-order structure includes points (e.g. point intensities) over one or more regions of the spectrum, such as the down field region (from about 6.5 ppm to about 10 ppm) and/or the upstream methyl regions (from about 0.5 ppm to about −1 ppm). In some embodiments, one or more regions of an NMR spectrum for analysis include from about −1.5 ppm to about 12 ppm, or about −1 ppm to about 1 ppm, or about 1 ppm to about 9 ppm, or about 0.5 ppm to about 6.5 ppm, or about 6.5 ppm to about 12 ppm, or about 9 ppm to about 12 ppm, or about 5 ppm to about 10 ppm, etc.

In some embodiments, representative regions of an NMR spectrum are quantified. For example, the point intensities over one or more regions of an NMR spectrum can be determined. In some instances, an NMR spectrum can be a 1D proton NMR spectrum comprising 65K or 128K points. In some embodiments, points (e.g. point intensities) over one or more regions of an NMR spectrum include 100-100,000 points, e.g., 1,000-50,000 points, 500-5,000 points, 1,000-10,000 points, etc.

In some instances, as described herein, shifts from a first state to a second state are obtained (e.g., after exposure to one or more stressors), and differences in signals associated with higher-order structure (e.g., before and after exposure to one or more stressors) are determined. Such differences can be obtained, for example, by quantifying one or more signals (e.g., peaks) from a first NMR spectrum obtained before exposure to a stressor, quantifying one or more signals (e.g., peaks) from a second NMR spectrum obtained after exposure to a stressor, and calculating a difference (a “delta”) between one or more quantified signals (e.g., relative peak intensities) from the first NMR spectrum and one or more corresponding quantified signals (e.g., relative peak intensities) from the second NMR spectrum.

In some embodiments, test protein deltas and target protein deltas are compared using one or more statistical analyses known in the art. For example, linear regression can be used to compare a test protein delta and a target protein delta. In some such methods, a correlation coefficient (R²) value can be determined to assess a level of similarity of, e.g., a protein before and after exposure to one or more stressors. In some instances, an R² value of greater than about 0.9 indicates a high level of similarity.

### Applications

In some instances, methods disclosed herein can be used to confirm the identity and/or quality of a protein, e.g., glycoprotein preparation. For example, methods can include assessing preparations (e.g., samples, lots, and/or batches) of a test protein, e.g., to confirm whether the test protein qualifies as a target protein, and, optionally, qualifying the test protein as a target protein if qualifying criteria (e.g. predefined qualifying criteria) are met; thereby evaluating, identifying, and/or producing (e.g., manufacturing) a protein product.

Methods of the disclosure have a variety of applications and include, e.g., quality control at different stages of manufacture, analysis of a protein preparation prior to and/or after completion of manufacture (e.g., prior to or after distribution to a fill/finish environment or facility), prior to or after release into commerce (e.g., before distribution to a pharmacy, a caregiver, a patient, or other end-user). In some instances, a protein preparation is a drug substance (an active pharmaceutical ingredient or “API”) or a drug product (an API formulated for use in a subject such as a human patient). In some instances, a protein preparation is from a stage of manufacture or use that is prior to release to care givers or other end-users; prior to packaging into individual dosage forms, such as syringes, pens, vials, or multi-dose vials; prior to determination that the batch can be commercially released, prior to production of a Certificate of Testing, Material Safety Data Sheet (MSDS) or Certificate of Analysis (CofA) of the preparation. In some instances, a protein preparation is from an intermediate step in production, e.g., it is after secretion of a protein from a cell but prior to purification of drug substance.

Evaluations from methods described herein are useful for guiding, controlling or implementing a number of activities or steps in the process of making, distributing, and monitoring and providing for the safe and efficacious use of a protein preparation. Thus, in an embodiment, e.g., responsive to the evaluation, e.g., depending on whether a criterion is met, a decision or step is taken. The method can further comprise one or both of the decision to take the step and/or carrying out the step itself. E.g., the step can comprise one in which the preparation (or another preparation for which the preparation is representative) is: classified; selected; accepted or discarded; released or processed into a drug product; rendered unusable for commercial release, e.g., by labeling it, sequestering it, or destroying it; passed on to a subsequent step in manufacture; reprocessed (e.g., the preparation may undergo a repetition of a previous process step or subjected to a corrective process); formulated, e.g., into drug substance or drug product; combined with another component, e.g., an excipient, buffer or diluent; disposed into a container; divided into smaller aliquots, e.g., unit doses, or multi-dose containers; combined with another preparation (e.g., another batch) of the protein; packaged; shipped; moved to a different location; combined with another element to form a kit; combined, e.g., placed into a package with a delivery device, diluent, or package insert; released into commerce; sold or offered for sale; delivered to a care giver or other end-user; or administered to a subject. E.g., based on the result of the determination or whether one or more subject entities is present, or upon comparison to a reference standard, the batch from which the preparation is taken can be processed, e.g., as just described.

Methods described herein may include making a decision: (a) as to whether a protein preparation may be formulated into drug substance or drug product; (b) as to whether a protein preparation may be reprocessed (e.g., the preparation may undergo a repetition of a previous process step); and/or (c) that the protein preparation is not suitable for formulation into drug substance or drug product. In some instances, methods comprise: formulating as referred to in step (a), reprocessing as referred to in step (b), or rendering the preparation unusable for commercial release, e.g., by labeling it or destroying it, as referred to in step (c).

### Test Proteins and Target Proteins

Methods described herein can be used to make and/or evaluate a test protein preparation, e.g., a test biologic preparation. In some embodiments, a “test protein” is a protein (e.g., a biologic) being evaluated for similarity to a target protein, e.g., a target biologic. A test biologic may or may not be commercially available. In some embodiments, a test biologic is not commercially available for therapeutic use in humans or animals. In some embodiments, a test biologic has not been approved for therapeutic or diagnostic use in humans or animals. In some embodiments, a test biologic has been approved, e.g., under a secondary approval process, for therapeutic or diagnostic use in humans or animals. In some embodiments, a test protein (e.g., test biologic) has the same primary amino acid sequence as a target protein (e.g., target biologic) or differs by no more than 1, 2, 3, 4, 5, 10, 15, 20, 25, 30 residues and/or has at least 80%, 85%, 90%, 91%, 92%, 93%, 94%, 95%, 96%, 97%, 98%, or 99% identity, or is 100% identical, to a target protein sequence (e.g., target biologic sequence). The terms the “same primary amino acid sequence”, “a primary amino acid sequence that differs by no more than 1, 2, 3, 4, 5, 10, 15, 20, 25, or 30 residues”, “sequences that have at least 98% or more sequence identity”, or similar terms, relate to level of identity between a primary amino acid sequence, e.g., of first protein, e.g., a test protein, and a primary amino acid sequence, e.g., of second protein, e.g., a target protein. In some embodiments, a protein preparation or product includes amino acid variants, e.g., species that differ at one or more terminal residues, e.g., at one or two terminal residues. In some embodiments of such cases, sequence identity compared is the identity between the primary amino acid sequence of the most abundant (e.g., most abundant active) species in each of the products being compared. In some embodiments, sequence identity refers to the amino acid sequence encoded by a nucleic acid that can be used to make the product.

In some instances, test proteins and target proteins described herein are antibodies, e.g., intact antibodies. As used herein, the term “antibody” refers to a polypeptide that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions.

The present methods can be used with antigen-binding fragments of antibodies (e.g., single chain antibodies, Fab, F(ab′)₂, Fd, Fv, and dAb fragments) as well as complete antibodies, e.g., intact immunoglobulins of types IgA, IgG, IgE, IgD, IgM (as well as subtypes thereof). The light chains of the immunoglobulin can be of types kappa or lambda. In some embodiments, an antibody includes an Fc region. In some embodiments, an antibody is a therapeutic antibody.

Antibodies described herein can include, for example, monoclonal antibodies, polyclonal antibodies (e.g., IVIG), multi specific antibodies, human antibodies, humanized antibodies, camelized antibodies, chimeric antibodies, single-chain Fvs (scFv), disulfide-linked Fvs (sdFv), and anti-idiotypic (anti-Id) antibodies, and antigen-binding fragments of any of the above. Antibodies can be of any type (e.g., IgG, IgE, IgM, IgD, IgA and IgY), class (e.g., IgG1, IgG2, IgG3, IgG4, IgA1 and IgA2) or subclass.

Antibodies or fragments thereof can be produced by any method known in the art for synthesizing antibodies (see, e.g., Harlow et al., Antibodies: A Laboratory Manual, (Cold Spring Harbor Laboratory Press, 2nd ed. 1988); Brinkman et al., 1995, J. Immunol. Methods 182:41-50; WO 92/22324; WO 98/46645). Chimeric antibodies can be produced using methods described in, e.g., Morrison, 1985, Science 229:1202, and humanized antibodies by methods described in, e.g., U.S. Pat. No. 6,180,370.

Nonlimiting, exemplary target antibodies can include abciximab (ReoPro®, Roche), adalimumab (Humira®, Bristol-Myers Squibb), alemtuzumab (Campath®, Genzyme/Bayer), basiliximab (Simulect®, Novartis), belimumab (Benlysta®, GlaxoSmithKline), bevacizumab (Avastin®, Roche), canakinumab (Ilaris®, Novartis), brentuximab vedotin (Adcetris®, Seattle Genetics), certolizumab (CIMZIA®, UCB, Brussels, Belgium), cetuximab (Erbitux®, Merck-Serono), daclizumab (Zenapax®, Hoffmann-La Roche), denosumab (Prolia®, Amgen; Xgeva®, Amgen), eculizumab (Soliris®, Alexion Pharmaceuticals), efalizumab (Raptiva®, Genentech), gemtuzumab (Mylotarg®, Pfizer), golimumab (Simponi®, Janssen), ibritumomab (Zevalin®, Spectrum Pharmaceuticals), infliximab (Remicade®, Centocor), ipilimumab (Yervoy™, Bristol-Myers Squibb), muromonab (Orthoclone OKT3®, Janssen-Cilag), natalizumab (Tysabri®, Biogen Idec, Elan), ofatumumab (Arzerra®, GlaxoSmithKline), omalizumab (Xolair®, Novartis), palivizumab (Synagis®, MedImmune), panitumumab (Vectibix®, Amgen), ranibizumab (Lucentis®, Genentech), rituximab (MabThera®, Roche), tocilizumab (Actemra®, Genentech; RoActemra, Hoffman-La Roche) tositumomab (Bexxar®, GlaxoSmithKline), trastuzumab (Herceptin®, Roche), and ustekinumab (Stelara®, Janssen).

### Recombinant Gene Expression

In accordance with the present disclosure, there may be employed conventional molecular biology, microbiology, and recombinant DNA techniques within the skill of the art. Such techniques are described in the literature (see, e.g., Sambrook, Fritsch & Maniatis, Molecular Cloning: A Laboratory Manual, Second Edition (1989) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells and Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

In some embodiments, a protein described herein is produced using recombinant methods. Recombinant expression of a gene, such as a gene encoding a polypeptide, such as an antibody described herein, can include construction of an expression vector containing a polynucleotide that encodes the polypeptide. Once a polynucleotide has been obtained, a vector for the production of the polypeptide can be produced by recombinant DNA technology using techniques known in the art. Known methods can be used to construct expression vectors containing polypeptide coding sequences and appropriate transcriptional and translational control signals. These methods include, for example, in vitro recombinant DNA techniques, synthetic techniques, and in vivo genetic recombination.

An expression vector can be transferred to a host cell by conventional techniques, and transfected cells can then be cultured by conventional techniques to produce polypeptide.

A variety of host expression vector systems can be used (see, e.g., U.S. Pat. No. 5,807,715). Such host-expression systems can be used to produce polypeptides and, where desired, subsequently purified. Such host expression systems include microorganisms such as bacteria (e.g., E. coli and B. subtilis) transformed with recombinant bacteriophage DNA, plasmid DNA or cosmid DNA expression vectors containing polypeptide coding sequences; yeast (e.g., Saccharomyces and Pichia) transformed with recombinant yeast expression vectors containing polypeptide coding sequences; insect cell systems infected with recombinant virus expression vectors (e.g., baculovirus) containing polypeptide coding sequences; plant cell systems infected with recombinant virus expression vectors (e.g., cauliflower mosaic virus, CaMV; tobacco mosaic virus, TMV) or transformed with recombinant plasmid expression vectors (e.g. Ti plasmid) containing polypeptide coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

For bacterial systems, a number of expression vectors can be used, including, but not limited to, the E. coli expression vector pUR278 (Ruther et al., 1983, EMBO 12:1791); pIN vectors (Inouye & Inouye, 1985, Nucleic Acids Res. 13:3101-3109; Van Heeke & Schuster, 1989, J. Biol. Chem. 24:5503-5509); and the like. pGEX vectors can also be used to express foreign polypeptides as fusion proteins with glutathione 5-transferase (GST).

For expression in mammalian host cells, viral-based expression systems can be utilized (see, e.g., Logan & Shenk, 1984, Proc. Natl. Acad. Sci. USA 8 1:355-359). The efficiency of expression can be enhanced by inclusion of appropriate transcription enhancer elements, transcription terminators, etc. (see, e.g., Bittner et al., 1987, Methods in Enzymol. 153:516-544).

In addition, a host cell strain can be chosen that modulates expression of inserted sequences, or modifies and processes the gene product in the specific fashion desired. Different host cells have characteristic and specific mechanisms for post-translational processing and modification of proteins and gene products. Appropriate cell lines or host systems can be chosen to ensure the correct modification and processing of the polypeptide expressed. Such cells include, for example, established mammalian cell lines and insect cell lines, animal cells, fungal cells, and yeast cells. Mammalian host cells include, but are not limited to, CHO, VERY, BHK, HeLa, COS, MDCK, 293, 3T3, W138, BT483, Hs578T, HTB2, BT20 and T47D, NS0 (a murine myeloma cell line that does not endogenously produce any immunoglobulin chains), CRL7O3O and HsS78Bst cells.

For long-term, high-yield production of recombinant proteins, host cells are engineered to stably express a polypeptide. Host cells can be transformed with DNA controlled by appropriate expression control elements known in the art, including promoter, enhancer, sequences, transcription terminators, polyadenylation sites, and selectable markers. Methods commonly known in the art of recombinant DNA technology can be used to select a desired recombinant clone.

Once a protein described herein been produced by recombinant expression, it may be purified by any method known in the art for purification, for example, by chromatography (e.g., ion exchange, affinity, and sizing column chromatography), centrifugation, differential solubility, or by any other standard technique for purification of proteins. For example, an antibody can be isolated and purified by appropriately selecting and combining affinity columns such as Protein A column with chromatography columns, filtration, ultra filtration, salting-out and dialysis procedures (see Antibodies: A Laboratory Manual, Ed Harlow, David Lane, Cold Spring Harbor Laboratory, 1988). Further, as described herein, a glycoprotein can be fused to heterologous polypeptide sequences to facilitate purification. Glycoproteins having desired sugar chains can be separated with a lectin column by methods known in the art (see, e.g., WO 02/30954).

### Pharmaceutical Compositions

A protein (e.g., an antibody) described herein can be incorporated into a pharmaceutical composition. Such a pharmaceutical composition is useful in the prevention and/or treatment of diseases. Pharmaceutical compositions comprising a polypeptide (e.g., an antibody) can be formulated by methods known to those skilled in the art (see, e.g., Remington's Pharmaceutical Sciences, 20th Ed., Lippincott Williams & Wilkins, 2000). The pharmaceutical composition can be administered parenterally in the form of an injectable formulation comprising a sterile solution or suspension in water or another pharmaceutically acceptable liquid. For example, the pharmaceutical composition can be formulated by suitably combining the polypeptide with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of active ingredient included in the pharmaceutical preparations is such that a suitable dose within the designated range is provided.

Route of administration can be parenteral, for example, administration by injection, transnasal administration, transpulmonary administration, or transcutaneous administration. Administration can be systemic or local by intravenous injection, intramuscular injection, intraperitoneal injection, subcutaneous injection.

A suitable means of administration can be selected based on the age and condition of the patient. A single dose of the pharmaceutical composition containing a polypeptide (e.g., antibody) can be selected from a range of 0.001 mg/kg of body weight to 1000 mg/kg of body weight. On the other hand, a dose can be selected in the range of 0.001 mg/kg of body weight to 100000 mg/kg of body weight, but the present disclosure is not limited to such ranges. The dose and method of administration varies depending on the weight, age, condition, and the like of the patient, and can be suitably selected as needed by those skilled in the art.

The disclosure is further illustrated by the following examples. The examples are provided for illustrative purposes only. They are not to be construed as limiting the scope or content of the disclosure in any way.

### EXAMPLES

### Example 1: Analysis of Biosimilarity of Intact Antibodies Using 2D NMR

2D HMQC NMR spectra were collected for different intact antibodies, and their peak composition and peak volumes were compared to assess biosimilarity.

Samples of an intact target antibody, an intact test antibody (having the same amino acid sequence as the target antibody), and two additional intact antibodies (“non-target antibody 1” and “non-target antibody 2”, each of which had amino acid sequences that differed from the target antibody) were buffer exchanged into a formulation buffer containing 7.34 mM Citrate, pH=5.2, 104 mM NaCl in 100% D₂O, at a concentration of 45-50 mg/mL. NMR samples were prepared in a regular NMR tube at 500 μL volume or in Shigemi tubes at 250 volume, and the experiments were acquired at 45° C. HMQC NMR acquisition time was around 2.5 days, with 2048×400 points acquired and 512 scans each point. Data was processed in Topspin software.

2D ¹H/¹³C HMQC experiment acquisition parameters are listed in Table 1:

TABLE 1

| | |
|---|---|
| Observe Nucleus | ¹H and ¹³C |
| Pulse Program | hmqcetgpsi |
| ¹H Pulse Width, P1 | 90° flip angle (at PLW1 power) |
| ¹³C Pulse Width, P3 | 90° flip angle, ~12 usec (at PLW2 power) |
| Transmitter offset, O1p | optimized for water suppression (~4.7 ppm) |
| Transmitter offset, O2p | ¹³C ~40 ppm |
| Sweep width ¹H Sweep | 12 ppm |
| width ¹³C* | 60 ppm (or less)* |
| Recycle delay, d1 | 1 second |
| Number of points, td | 2K |
| Number of t1 points | 400 or less |
| Number of scans, ns | 512 or less |
| Temperature | 45° C. |

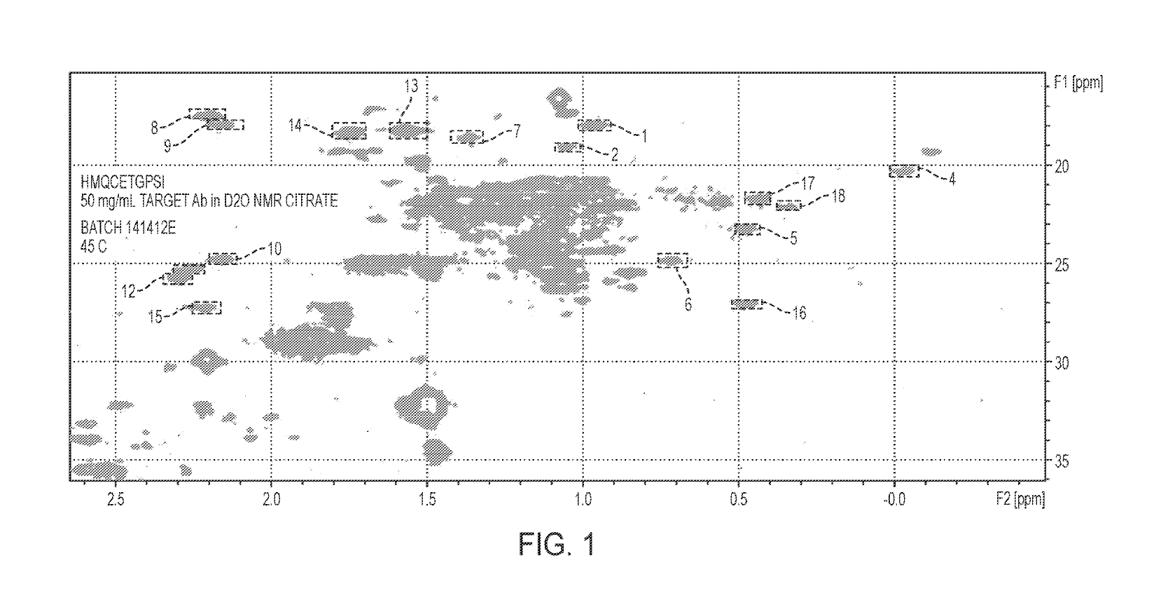

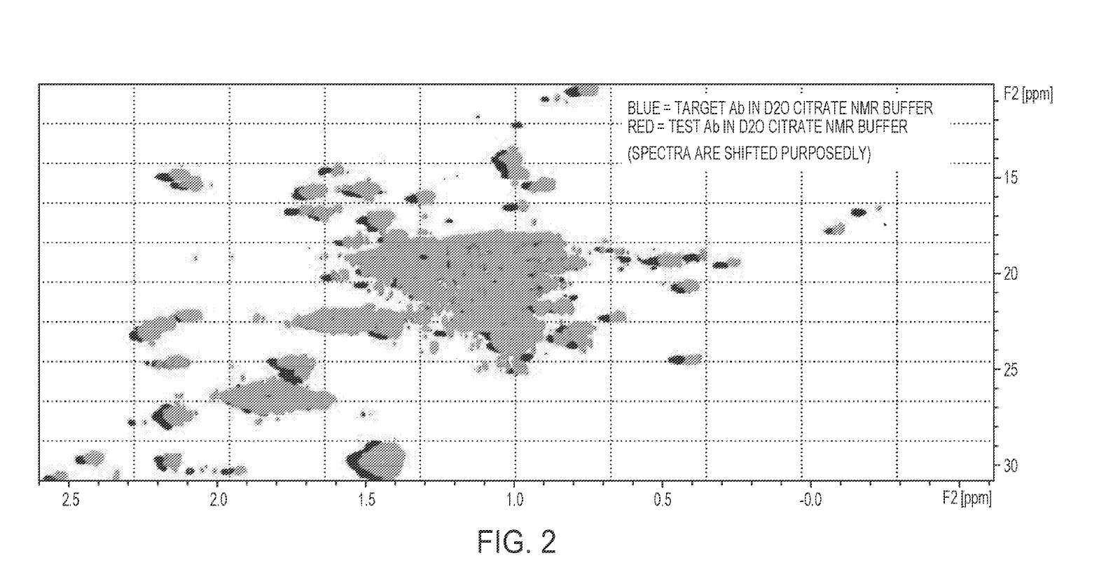

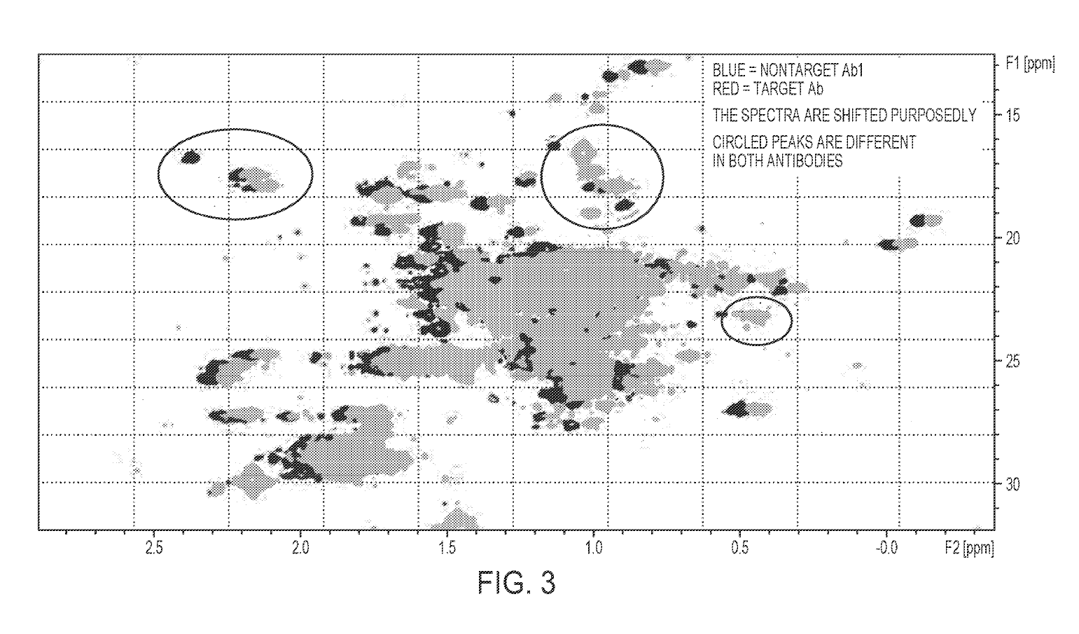

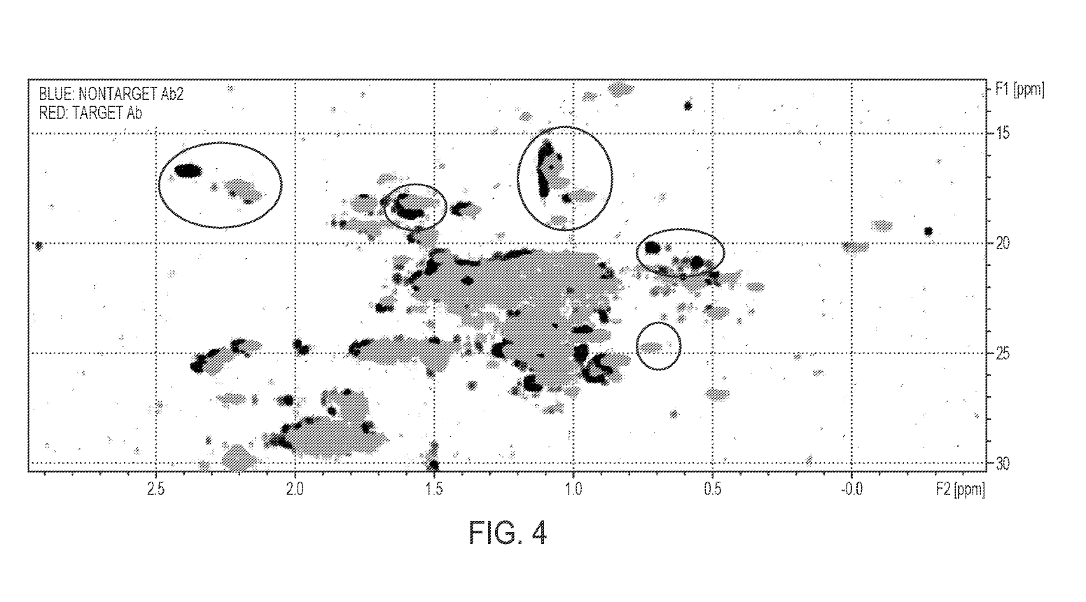

The 2D HMQC spectrum of glycosylated target antibody (where glycans were cleaved from the target antibody), showing methyl peaks, is depicted in FIG. 1. The overlays of 2D HMQC NMR spectra of the target antibody with the test antibody are shown in FIG. 2 (which shows methyl peaks). Spectral similarities were observed for methyl peaks in the target antibody and the test antibody, as shown in FIG. 2. Overlays of 2D HMQC NMR spectra of the target antibody with non-target antibody 1 is shown in FIG. 3; and spectra of the target antibody with non-target antibody 2 is shown in FIG. 4 (all figures depicting methyl peaks), where the differences are highlighted.

Eighteen peaks in the methyl region were chosen from each Figure and integrated to generate peak volumes as shown in Table 2:

TABLE 2
Comparison of peak volumes:

| | Target | Test | Non-target | Non-target |
|---|---|---|---|---|
| Peaks | Antibody | Antibody | Antibody 1 | Antibody 2 |
| Integral 1 | 1.00 | 1.00 | 1.00 | 1.00 |
| Integral 2 | 0.33 | 0.36 | 0.01 | 0.01 |
| Integral 3 | 1.18 | 1.07 | 1.11 | 1.10 |
| Integral 4 | 0.48 | 0.47 | 0.51 | 0.42 |
| Integral 5 | 0.62 | 0.66 | 0.22 | 0.22 |
| Integral 6 | 0.59 | 0.65 | 0.05 | 0.05 |
| Integral 7 | 0.82 | 0.79 | 0.85 | 1.08 |
| Integral 8 | 1.05 | 0.97 | 0.97 | 1.01 |
| Integral 9 | 0.80 | 0.77 | 0.65 | 0.71 |
| Integral 10 | 0.77 | 0.75 | 0.77 | 0.70 |
| Integral 11 | 1.00 | 1.03 | 1.01 | 1.29 |
| Integral 12 | 1.35 | 1.31 | 1.37 | 1.70 |
| Integral 13 | 2.08 | 2.03 | 2.15 | 2.11 |
| Integral 14 | 1.20 | 1.17 | 0.84 | 0.81 |
| Integral 15 | 0.51 | 0.56 | 0.61 | 0.49 |
| Integral 16 | 0.51 | 0.53 | 0.60 | 0.37 |
| Integral 17 | 0.53 | 0.56 | 0.37 | 0.28 |
| Integral 18 | 0.32 | 0.34 | 0.28 | 0.17 |

Initial analysis was performed where the R² coefficients from pairwise comparisons were generated. Correlation plots of peak volumes for pairs of antibodies were produced, and the R² coefficient of the corresponding plot was calculated. Specifically, a linear regression analysis was performed for each point, and the goodness of fit (expressed as R²) was obtained. For identical datasets, R²=1, and lower values of R² denote that the datasets deviate from identity. The results are depicted in Table 3:

TABLE 3
R² coefficient of fit

| Sample | R² |
|---|---|
| Target antibody vs Test antibody | 0.947 |
| Target antibody vs Non-target antibody 1 | 0.8734 |
| Target antibody vs Non-target antibody 2 | 0.8547 |
| Target antibody vs Deglycosylated Target antibody | 0.4554 |
| Test antibody vs Test antibody* | 0.974 |

*Same sample of test antibody was collected back to back

An R² value of 0.947 was calculated when comparing the target antibody and the test antibody, and an R² value of 0.974 was calculated when comparing the test antibody to itself. Comparisons of the target antibody to either non-target antibody 1 or non-target antibody 2 resulted in an R² value of 0.8734 and 0.8547, respectively.

In summary, 2D HMQC NMR resolved methyl peaks for different intact antibodies, providing a “signature” spectrum for each type of antibody, without requiring digestion or labeling. The peak position and peak volume were highly dependent on antibody sequence and folding, demonstrating the high sensitivity of the method to three dimensional structure.

### Example 2: Characterization of Antibodies Using Stressors

Samples of an intact target antibody, two intact test antibodies (each having the same amino acid sequence as the target antibody), and two additional intact antibodies (“non-target antibody 1” and “non-target antibody 2”, each of which had amino acid sequences that differed from the target antibody) were buffer exchanged into a formulation buffer containing 7.34 mM Citrate, pH=5.2, 104 mM NaCl in 10% D₂O/90% H₂O or in 100% D₂O.

For sample preparation, 10× stock solutions of 1-5 mM DSS-d6 were prepared by dissolving 2.3-11.5 mg in 10 mL of NMR citrate buffer. A 7.5 mM solution of TbCl₃ DMSO-d6/D₂O was prepared by dissolving 9.95 mg TbCl₃ in 0.5 mL DMSO-d6 and 45 mL D₂O buffer. A solution of 90 mM DPA DMSO-d6 was prepared by dissolving 15.0 mg DPA in 1 mL of DMSO-d6. To prepare [Tb(DPA)₃]³⁻ complex solution, 2 mL of 7.5 mM TbCl₃, 0.5 mL of 90 mM DPA, and 2.5 mL D₂O were combined and mixed. A 1 mL stock solution of 300 mM TempoL was prepared by dissolving 51.6 mg of TempoL in 1 mL citrate H₂O/D₂O NMR buffer solution, and stored at room temperature.

For 1D Proton NMR at 35° C., 1D proton at 55° C. and 2D ¹H-¹³C HMQC (described in Example 1), target protein NMR samples were prepared by taking 450 μL of target protein in its original buffer directly from the syringe and adding 50 μL of 10×DSS-d6 stock solution. For 1D Proton NMR at 55° C. in 80% D₂O, 100 μL of target protein was taken from the syringe and mixed with 400 μL D₂O and 50 μL of 10×DSS-d6 stock solution.

1D Proton NMR acquisition parameters are listed in Table 4:

TABLE 4

| | |
|---|---|
| Observe Nucleus | ¹H |
| Pulse Program | zgespg |
| ¹H Pulse Width, P1 | 90° flip angle |
| Transmitter offset, O1p | optimized for water suppression (~4.7 ppm) |
| Sweep Width | 16 ppm |
| Recycle delay, d1 | 1 second |
| Number of points, td | 32K |
| Number of scans, ns | 1024 or less |
| Water suppression, | Optimized for water suppression |
| SPDB1 | |
| Temperature | 35° C. or 55° C. |

All samples were analyzed following exposure to the following stressors: different temperatures (35° C. and 55° C.), Tb⁺³, TempoL, and D₂O exchange (50% D₂O/35° C.; 50% D₂O/55° C.; 80% D₂O/35° C.; and 80% D₂O/55° C.). NMR spectra were obtained for the samples in the presence of each stressor, and relative peak intensity was determined for each sample and condition. Peak intensities were obtained for 20 peaks across the 1D spectrum at the following chemical shifts:

| | Chemical |
|---|---|
| Peak | shift (ppm) |
| 1 | 8.44 |
| 2 | 8.35 |
| 3 | 8.24 |
| 4 | 8.18 |
| 5 | 7.95 |
| 6 | 7.80 |
| 7 | 7.60 |
| 8 | 7.52 |
| 9 | 6.97 |
| 10 | 7.19 |
| 11 | 6.94 |
| 12 | 6.79 |
| 13 | 6.68 |
| 14 | 0.81 |
| 15 | 0.76 |
| 16 | 0.57 |
| 17 | 0.38 |
| 18 | 0.17 |
| 19 | ~0.17 |
| 20 | ~0.31 |

Peak intensities were all normalized to the intensity of Peak 14. Examples of RPI (Relative Peak Intensity) for different samples under different conditions:

| | | | | | | Target Ab | Nontarget | Nontarget |
|---|---|---|---|---|---|---|---|---|
| | Chemical | Target Ab | Target Ab | Target Ab | Target Ab | 80% D₂O | Ab 1 | Ab 2 |
| Peak | shift (ppm)1 | 35° C. | 55° C. | with Tb + 3 | with TempoL | 55° C. | 35° C. | 35° C. |
| 1 | 8.44 | 0.22 | 0.17 | 0.15 | 0.21 | 0.12 | 0.19 | 0.23 |
| 2 | 8.35 | 0.15 | 0.17 | 0.15 | 0.15 | 0.07 | 0.12 | 0.15 |
| 3 | 8.24 | 0.16 | 0.18 | 0.15 | 0.14 | 0.10 | 0.13 | 0.17 |
| 4 | 8.18 | 0.18 | 0.14 | 0.16 | 0.17 | 0.08 | 0.14 | 0.19 |
| 5 | 7.95 | 0.19 | 0.16 | 0.19 | 0.16 | 0.07 | 0.17 | 0.22 |
| 6 | 7.80 | 0.15 | 0.18 | 0.12 | 0.15 | 0.06 | 0.11 | 0.14 |
| 7 | 7.60 | 0.28 | 0.15 | 0.22 | 0.26 | 0.08 | 0.26 | 0.31 |
| 8 | 7.52 | 0.29 | 0.19 | 0.25 | 0.29 | 0.10 | 0.27 | 0.32 |
| 9 | 6.97 | 0.42 | 0.36 | 0.33 | 0.41 | 0.31 | 0.39 | 0.41 |
| 10 | 7.19 | 0.50 | 0.36 | 0.37 | 0.43 | 0.35 | 0.41 | 0.49 |
| 11 | 6.94 | 0.54 | 0.48 | 0.44 | 0.44 | 0.47 | 0.61 | 0.58 |
| 12 | 6.79 | 0.58 | 0.43 | 0.40 | 0.47 | 0.34 | 0.37 | 0.48 |
| 13 | 6.68 | 0.31 | 0.36 | 0.25 | 0.27 | 0.29 | 0.29 | 0.38 |
| 14 | 0.81 | 1.00 | 1.00 | 1.00 | 1.00 | 1.00 | 1.00 | 1.00 |
| 15 | 0.76 | 0.83 | 0.78 | 0.86 | 0.81 | 0.77 | 0.77 | 0.92 |
| 16 | 0.57 | 0.49 | 0.51 | 0.44 | 0.47 | 0.50 | 0.38 | 0.42 |
| 17 | 0.38 | 0.34 | 0.29 | 0.35 | 0.34 | 0.26 | 0.37 | 0.37 |
| 18 | 0.17 | 0.22 | 0.17 | 0.22 | 0.22 | 0.17 | 0.16 | 0.18 |
| 19 | −0.17 | 0.10 | 0.04 | 0.11 | 0.11 | 0.04 | 0.08 | 0.10 |
| 20 | −0.31 | 0.08 | 0.03 | 0.08 | 0.09 | 0.03 | 0.08 | 0.09 |

1Values may vary by +/−0.03 ppm

For data analysis, the intensity of peak 14 was set to 1.00 and relative peak intensities (RPI) for the remaining nineteen peaks were calculated. The peak intensities were compared between the test protein sample and target protein sample by generating a correlation R² in excel. The target protein sample was plotted the in the X axis and the test sample in the Y axis for the R² correlation plots. Data was also analyzed by comparing the ΔRPI values, which corresponds to the difference between [RPI in perturbed (stressed) state−RPI in unperturbed (unstressed) state].

Temperature

Temperature as a stressor was assessed using 1D Proton NMR at 35° C. and 1D Proton NMR at 55° C. For this analysis, ΔRPI=[RPI at 55° C.−RPI at 35° C.].

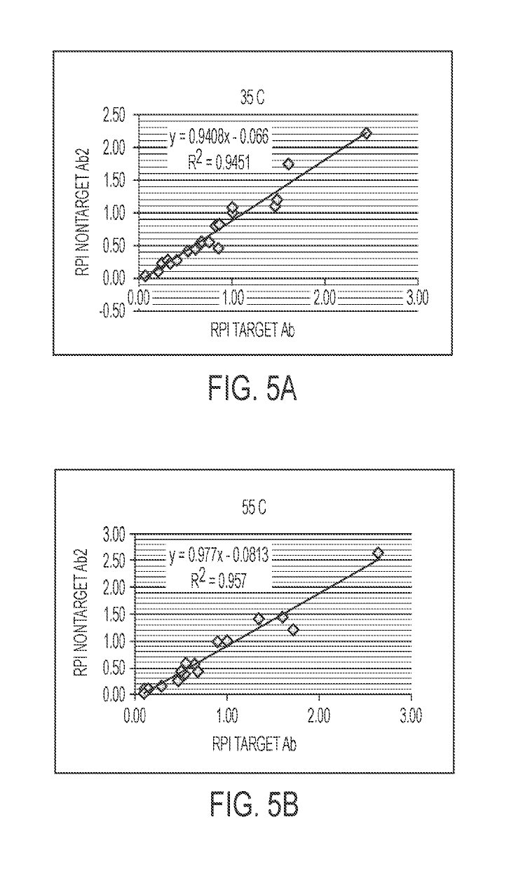

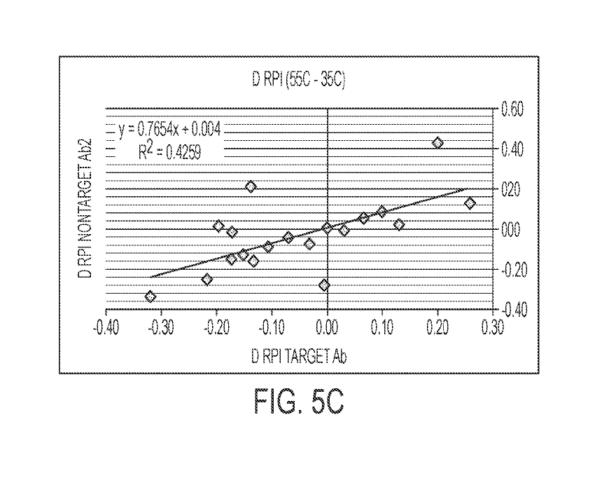

FIG. 5A depicts linear regression analysis of a comparison of relative peak intensity of the target antibody versus non-target antibody 2 at 35° C. FIG. 5B depicts linear regression analysis of a comparison of relative peak intensity of the target antibody versus non-target antibody 2 at 55° C. As indicated, at 35° C., an R² value of 0.9451 was calculated, and at 55° C., an R² value of 0.957 was calculated. Thus, even though the target antibody and non-target antibody 2 differed in amino acid sequence, no differences in relative peak intensity for the target antibody versus non-target antibody 2 at 35° C. or at 55° C. were seen. Surprisingly, when ΔRPI were calculated (i.e., RPI at 55° C.−RPI at 35° C.) and the difference in relative peak intensity subjected to linear regression analysis, an R² value of 0.4259 was obtained (as shown in FIG. 5C). This demonstrates that differences in relative peak intensity can be used to assess similarity of higher-order structure of proteins.

Table 5 summarizes the R² values calculated for pairs of samples:

TABLE 5

| Samples | R² |
|---|---|
| 35 C. | |
| Target Ab + Test Ab 1 | 0.9994 |
| Target Ab + Test Ab 2 | 0.9992 |
| Target Ab + Test Ab2* | 0.9974 |
| Target Ab + Nontarg Ab1 | 0.9574 |
| Target Ab + Nontarg Ab2 | 0.9451 |
| 55 C. | |
| Target Ab + Test Ab1 | 0.9965 |
| Target Ab + Test Ab2 | 0.9972 |
| Target Ab + Test Ab2* | 0.9956 |
| Target Ab + Nontarg Ab1 | 0.9095 |
| Target Ab + Nontarg Ab2 | 0.9570 |
| Δ RPI 55 C.-35 C. | |
| Test Ab1 + Test Ab2 | 0.9972 |
| Target Ab + Test Ab1 | 0.9123 |
| Target Ab + Test Ab2 | 0.9207 |
| Target Ab + Target Ab2* | 0.8945 |
| Target Ab + Nontarg Ab1 | 0.1542 |
| Target Ab + Nontarg Ab2 | 0.4259 |

*Target Ab2 = Target Ab with 2x tween/mannitol

Tb⁺³ Shift Agent

Tb+3 complex as a stressor was assessed using 1D Proton NMR at 35° C. with addition of Tb+3 complex. For this analysis, ΔRPI=[RPI with Tb at 35° C.−RPI at 35° C.].

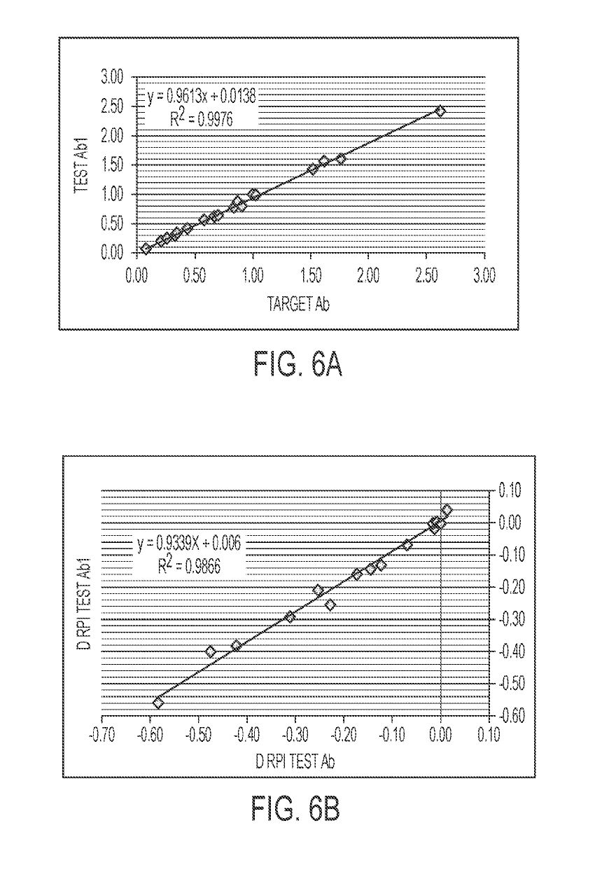

FIG. 6A depicts linear regression analysis of a comparison of relative peak intensity of the target antibody versus test antibody 1 in the presence of the Tb⁺³ shift agent. As indicated in FIG. 6A, an R² value of 0.9976 was calculated. FIG. 6B depicts linear regression analysis of differences in relative peak intensities between the presence and the absence of the Tb⁺³ shift agent, indicating an R² value of 0.9866. Table 6 summarizes the R² values calculated for pairs of samples:

TABLE 6

| Sample | R² | Δ RPI R² |
|---|---|---|
| Tb + 3 | | |
| Test Ab1 + Test Ab2 | 0.9999 | 0.9938 |
| Target Ab + Test Ab1 | 0.9976 | 0.9866 |
| Target Ab + Test Ab2* | 0.9988 | 0.9896 |
| Target Ab + Nontarg 1 | 0.9528 | 0.2271 |
| Target Ab + Nontarg 2 | 0.9468 | 0.2896 |

TempoL Shift Agent

TempoL as a stressor was assessed using 1D Proton NMR at 35° C. with addition of TempoL. For this analysis, ΔRPI=[RPI with TempoL at 35° C.−RPI at 35° C.].

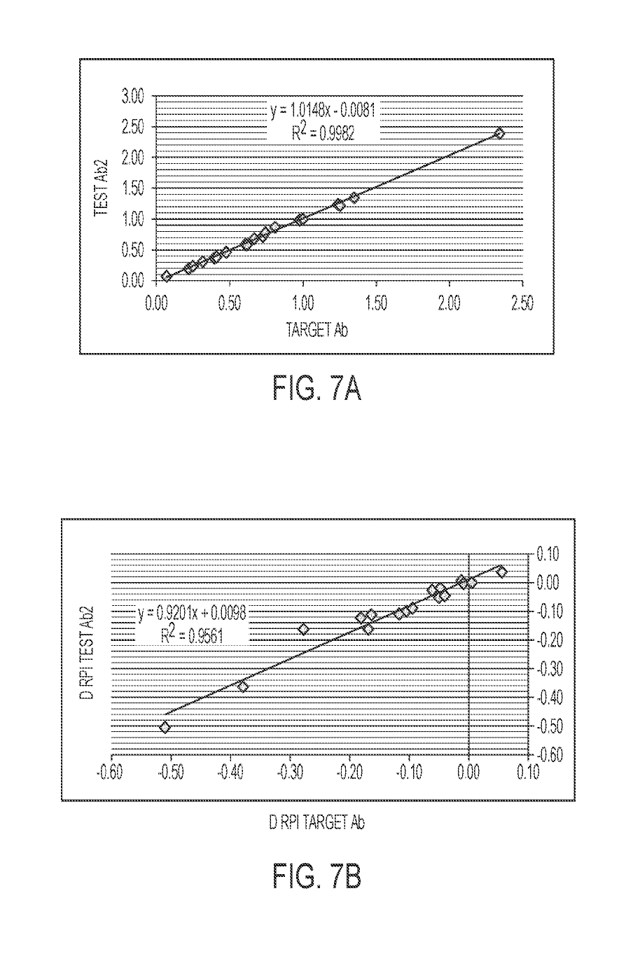

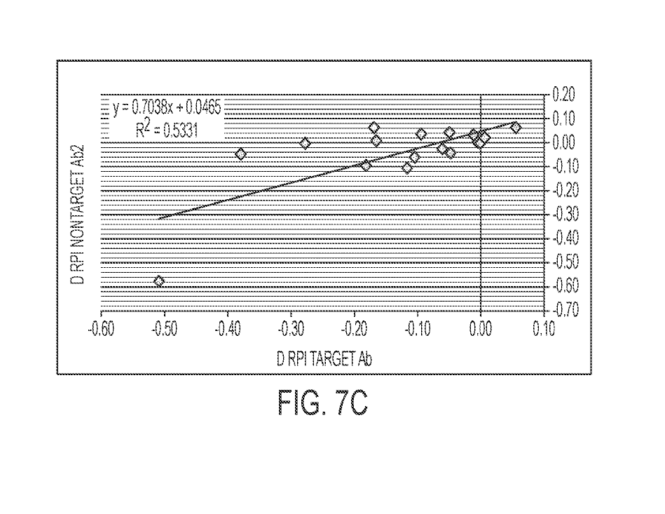

FIG. 7A depicts linear regression analysis of a comparison of relative peak intensity of the target antibody versus test antibody 2 in the presence of TempoL. As indicated in FIG. 7A, an R² value of 0.9982 was calculated. FIG. 7B depicts linear regression analysis of differences in relative peak intensities between the presence of TempoL and the absence of TempoL, for the target antibody versus test antibody 2, indicating an R² value of 0.9561. FIG. 7C illustrates linear regression analysis of differences in relative peak intensities between the presence of TempoL and the absence of TempoL, for the target antibody versus non-target antibody 2, indicating an R² value of 0.5331. Table 7 summarizes the R² values calculated for pairs of samples:

TABLE 7

| Sample | R² | Δ RPI R² |
|---|---|---|
| TempoL | | |
| Test Ab1 + Test Ab2 | 0.9993 | 0.9952 |
| Target Ab + Test Ab1 | 0.9984 | 0.9491 |
| Target Ab + Test Ab2* | 0.9982 | 0.9561 |
| Target Ab + Nontarg 1 | 0.9787 | 0.1050 |
| Target Ab + Nontarg 2 | 0.9825 | 0.5331 |

D₂O Exchange

D₂O as a stressor was assessed using 1D Proton NMR at 55° C. with D₂O exchange. For this analysis, ΔRPI=[RPI with D₂O at 55° C.−RPI at 55° C.].

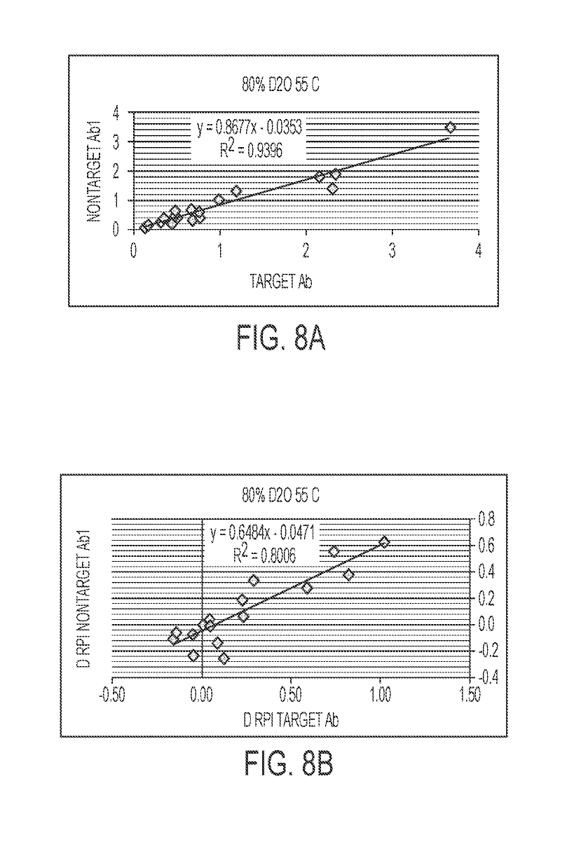

FIG. 8A depicts linear regression analysis of a comparison of relative peak intensity of the target antibody versus non-target antibody 1 at 80% D₂O at 55° C. As indicated in FIG. 8A, an R² value of 0.9396 was calculated. FIG. 8B depicts linear regression analysis of differences in relative peak intensities between the presence of 80% D₂O at 55° C. and the absence of this stressor, for the target antibody versus non-target antibody 1, indicating an R² value of 0.8006. Table 8 summarizes the R² values calculated for pairs of samples:

TABLE 8

| 80% D2O 55 C. | |
|---|---|
| Target Ab + Target Ab* | 0.9955 |
| Target Ab + Target Ab** | 0.9974 |
| Target Ab + Test Ab1 | 0.998 |
| Target Ab + Test Ab2 | 0.9933/0.9917 |
| Target Ab + Nontarg Ab2 | 0.9532 |
| Target Ab + Nontarg Ab1 | 0.9396 |
| 80% D2O 55 C. | Δ RPI R² |
| Target Ab + Target Ab* | 0.9642 |
| Target Ab + Target Ab** | 0.9799 |
| Target Ab + Test Ab1 | 0.976 |
| Test Ab1 + Test Ab2 | 0.9642 |
| Target Ab + Test Ab2 | 0.9508 |
| Target Ab + Nontarg Ab2 | 0.8888 |
| Target Ab + Nontarg Ab1 | 0.8006 |

*= Two different lots
**= Two different commercial sources

### Example 3: Characterization of Antibodies by Comparison of Point Intensities

1D Proton NMR spectra were collected for different intact antibodies, and point intensity over a region of the spectrum were compared to assess biosimilarity. Samples of an intact target antibody, an intact test antibody (having the same amino acid sequence as the target antibody) and two additional intact antibodies (referred to herein as “non-target antibody 1” and “non-target antibody 2”) were analyzed. All samples were analyzed following exposure to the following stressors: different temperatures (35° C. and 55° C.), Tb⁺³ and TempoL. Sample preparation for 1D Proton NMR and acquisition parameters were as described in Example 2.

For all samples, point intensities were evaluated from 6.49 ppm to 12.00 ppm, using 2259 points. To decrease the number of points for comparison, point intensities were binned by averaging the intensity for every 10 points. Comparison between two samples was made by plotting a correlation plot, where all the points/intensities for both samples are plotted and the correlation of fit is calculated.

Temperature

For antibody samples analyzed at 35° C. and 55° C., correlation plots of point intensities for pairs of antibodies were produced, and the R² coefficient of the corresponding plot was calculated. Specifically, a linear regression analysis was performed, and the goodness of fit (expressed as R²) was obtained.

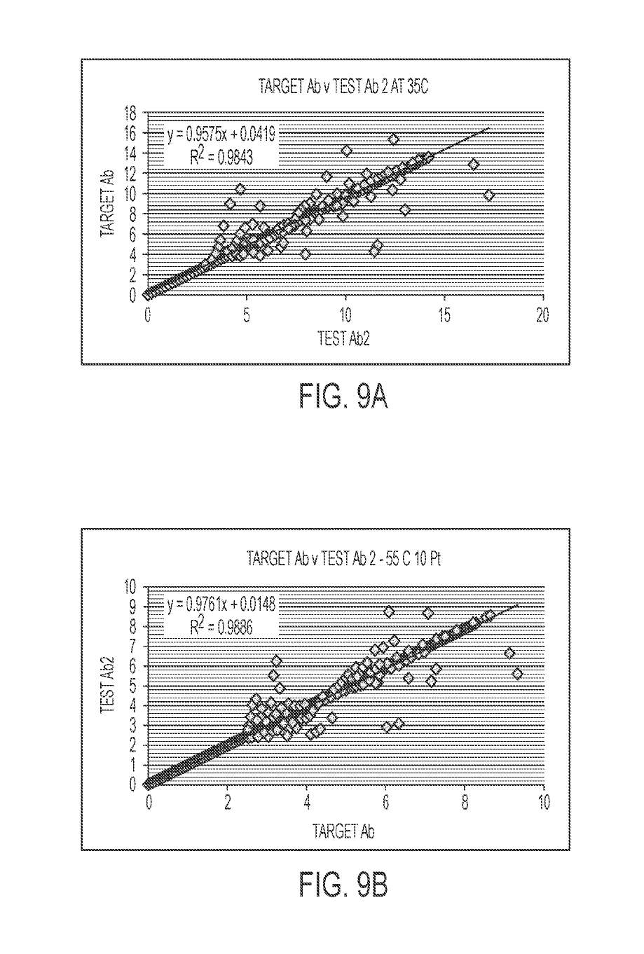

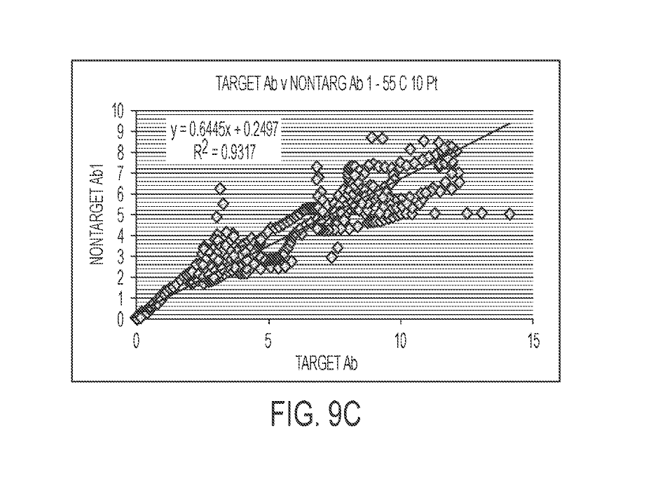

As shown in FIGS. 9A and 9B, samples of an intact target antibody and an intact test antibody show strong correlation of fit when analyzed at either 35° C. or 55° C. FIG. 9A depicts linear regression analysis of a comparison of point intensities from 6.49 ppm to 12.00 ppm of the target antibody versus test antibody 1 at 35° C., with a calculated R² value of 0.9843. FIG. 9B depicts the linear regression analysis point intensities from 6.49 ppm to 12.00 ppm of the target antibody versus test antibody 2 at 55° C., with a calculated R² value of 0.9886.

A reduced correlation of fit was observed for analysis of point intensities for target antibody versus non-target antibody. FIG. 9C depicts the linear regression analysis point intensities from 6.49 ppm to 12.00 ppm of the target antibody versus non-target antibody 1 at 55° C., with a calculated R² value of 0.9317. For identical datasets, R²=1, and lower values of R² denote that the datasets deviate from identity. Tables 9 and 10 below summarize the R² values calculated for all pairs of samples analyzed at 35° C. and 55° C., respectively.

TABLE 9
R² coefficient of fit at 35° C.:

| | Nontarg | Nontarg | | | |
|---|---|---|---|---|---|
| Sample | Ab1 | Ab2 | Test Ab_1 | Test Ab_2 | Target Ab |
| Nontarg Ab1 | 1.0000 | 0.9269 | 0.9066 | 0.9071 | 0.9017 |
| Nontarg Ab2 | 0.9269 | 1.0000 | 0.9521 | 0.9504 | 0.9631 |
| Test Ab_1 | 0.9066 | 0.9521 | 1.0000 | 0.9991 | 0.9874 |
| Test Ab_2 | 0.9071 | 0.9504 | 0.9991 | 1.0000 | 0.9843 |
| Target Ab | 0.9017 | 0.9631 | 0.9874 | 0.9843 | 1.0000 |

TABLE 10
R² coefficient of fit at 55° C.:

| | Nontarg | Nontarg | | | |
|---|---|---|---|---|---|
| Sample | Ab1 | Ab2 | Test Ab_1 | Test Ab_2 | Target Ab |
| Nontarg Ab1 | 1.0000 | 0.9513 | 0.9292 | 0.9313 | 0.9317 |
| Nontarg Ab2 | 0.9513 | 1.0000 | 0.9532 | 0.9535 | 0.9558 |
| Test Ab_1 | 0.9292 | 0.9532 | 1.0000 | 0.9981 | 0.9912 |
| Test Ab_2 | 0.9313 | 0.9535 | 0.9981 | 1.0000 | 0.9886 |
| Target Ab | 0.9317 | 0.9558 | 0.9912 | 0.9886 | 1.0000 |

Tb⁺³ Shift Agent

Analysis of point intensities of samples treated with Tb⁺³ show a strong correlation of fit between target antibody and test antibody, and a reduced correlation of fit for analysis of target antibody versus non-target antibody. For antibody samples treated with Tb⁺³, R² coefficients from pairwise comparisons were generated. Correlation plots of point intensities for pairs of antibodies were produced, and the R² coefficient of the corresponding plot was calculated. Specifically, a linear regression analysis was performed, and the goodness of fit (expressed as R²) was obtained. For identical datasets, R²=1, and lower values of R² denote that the datasets deviate from identity. The results are depicted in Table 11:

TABLE 11
R² coefficient of fit of sample treated with Tb⁺³:

| | Nontarg | Nontarg | | | |
|---|---|---|---|---|---|
| Sample | Ab1 | Ab2 | Test Ab_1 | Test Ab_2 | Target Ab |
| Nontarg Ab1 | 1.0000 | 0.9759 | 0.9661 | 0.9683 | 0.9652 |
| Nontarg Ab2 | 0.9759 | 1.0000 | 0.9788 | 0.9801 | 0.9763 |
| Test Ab_1 | 0.9661 | 0.9788 | 1.0000 | 0.9995 | 0.9986 |
| Test Ab_2 | 0.9683 | 0.9801 | 0.9995 | 1.0000 | 0.9990 |
| Target Ab | 0.9652 | 0.9763 | 0.9986 | 0.9990 | 1.0000 |

TempoL Shift Agent

Analysis of point intensities of samples exposed to TempoL also show a strong correlation of fit between target antibody and test antibody, and a reduced correlation of fit for analysis of target antibody versus non-target antibody. For antibody samples treated with TempoL, R² coefficients from pairwise comparisons were generated. Correlation plots of point intensities for pairs of antibodies were produced, and the R² coefficient of the corresponding plot was calculated. Specifically, a linear regression analysis was performed, and the goodness of fit (expressed as R²) was obtained. For identical datasets, R²=1, and lower values of R² denote that the datasets deviate from identity. The results are depicted in Table 12:

TABLE 12
R² coefficient of fit of sample treated with TempoL:

| | | | Test | Test | Target |
|---|---|---|---|---|---|
| Sample | Nontarg Ab1 | Nontarg Ab2 | Ab_1 | Ab_2 | Ab |
| Nontarg_Ab1 | 1.0000 | 0.9771 | 0.9726 | 0.9736 | 0.9632 |
| Nontarg_Ab2 | 0.9771 | 1.0000 | 0.9786 | 0.9814 | 0.9740 |
| Test Ab_1 | 0.9726 | 0.9786 | 1.0000 | 0.9986 | 0.9971 |
| Test Ab_2 | 0.9736 | 0.9814 | 0.9986 | 1.0000 | 0.9944 |
| Target Ab | 0.9632 | 0.9740 | 0.9971 | 0.9944 | 1.0000 |

In summary, analysis of point intensities of 1D proton NMR spectra for intact antibodies under different conditions (35° C., 55° C. and exposure to Tb⁺³, TempoL) showed strong correlation of fit (R²) between target antibody and test antibody and a reduced correlation of fit (R²) between target antibody and non-target antibody. Thus, analysis of point intensities of 1D proton NMR spectra can also be used to assess biosimilarity of antibodies.

### Example 4: Manufacture of a Biosimilar Protein

Samples of an intact test protein are obtained, which protein is in a first state. A sample of the test protein in the first state is exposed to a stressor to obtain a sample of the test protein in a second state. NMR is used to detect representative peaks for the protein in the first state and corresponding peaks for the protein in the second state. Differences in relative peak intensities are determined between the representative peaks for the protein in the first state and corresponding peaks for the protein in the second state to determine a test protein delta. Linear regression analysis is used to compare the test protein delta to a corresponding target protein delta of a target protein to produce a linear regression plot. The target protein has an amino acid sequence at least 98% identical to the test protein. An R² value of 0.91 is determined for the linear regression plot, which is tolerable. The test protein is processed into drug product for administration.

### EQUIVALENTS

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of manufacture comprising:
producing a batch of test protein drug substance;
exposing a sample of the batch of the test protein in a first state to a plurality of stressors to obtain a plurality of test protein in a second state, wherein one or more of the plurality of stressors comprises a condition that alters a higher-order structure of a protein;
detecting a signal associated with higher-order structure of the test protein for each of the plurality of test protein in the second state, wherein the detecting comprises use of an NMR method;
determining a test protein delta between a signal associated with higher-order structure of the test protein drug product in the first state and the signal associated with higher-order for each of the plurality of test protein drug product in the second state;
comparing the determined test protein deltas to corresponding target protein deltas of a target protein drug product approved under a primary approval process to determine whether the test protein deltas and the target protein deltas are tolerable; and
processing the batch of the test protein drug substance as test protein drug product if the test protein deltas and the target protein deltas are tolerable, or
taking an alternative action if the test protein deltas and the target protein deltas are not tolerable.

2. (canceled)

3. The method of claim 1, wherein the first state is a native state and the second state is a non-native state.

4. (canceled)

5. The method of claim 1, wherein one or more of the plurality of stressors comprises an NMR shift reagent.

6. (canceled)

7. The method of claim 1, wherein the compared test protein deltas and target protein deltas are tolerable if they meet a predetermined value.

8. The method of claim 1, wherein the signal associated with higher-order structure comprises one or more peaks from an NMR spectrum.

9. The method of claim 1, wherein the alternative action comprises one or more of disposing of the batch of the test protein drug substance, classifying for disposal the batch of the test protein drug substance, labeling the batch of the test protein drug substance for disposal, and reprocessing the batch of the test protein drug substance.

10. The method of claim 1, further comprising detecting a signal associated with higher-order structure of the test protein drug substance in the first state.

11. The method of claim 1, wherein the comparing step comprises producing a representation of the comparison of the test protein deltas and the target protein deltas.

12. The method of claim 1, wherein the target protein has an amino acid sequence at least 98% identical to the test protein, and wherein the target protein is approved under a BLA.

13.-23. (canceled)

24. A method of manufacture comprising:
producing a batch of a test protein drug substance, wherein the test protein drug substance is not approved under a BLA;
exposing a sample of the test protein drug substance in a first state to a stressor to obtain a test protein drug substance in a second state, wherein the stressor comprises a condition that alters a higher-order structure of a protein;
detecting a signal associated with higher-order structure of the test protein drug substance in the second state, wherein detecting a signal comprises an NMR method;
determining a test protein delta between a signal associated with higher-order structure of the test protein drug substance in the first state and the signal associated with higher-order for the test protein drug substance in the second state;
comparing the determined test protein delta to a corresponding target protein delta of a target protein drug product to determine whether the test protein delta and the target protein delta are tolerable; and
processing the batch of the test protein drug substance as drug product if the test protein delta and the target protein delta are tolerable; or
taking an alternative action if the test protein delta and the target protein delta are not tolerable.

25.-32. (canceled)

33. The method of claim 24, further comprising detecting a signal associated with higher-order structure of the test protein drug substance in the first state.

34. The method of claim 24, wherein the comparing step comprises producing a representation of the comparison of the test protein deltas and the target protein deltas.

35.-47. (canceled)

48. A method of manufacture, comprising:
providing a first preparation of an intact test glycoprotein drug substance, wherein the test glycoprotein drug product is not approved under a BLA;
obtaining a first 2D NMR spectrum of the first preparation;
providing a second preparation of an intact target glycoprotein drug product, wherein the target glycoprotein has an amino acid sequence at least 98% identical to the test glycoprotein, and wherein the target glycoprotein drug product is approved under a BLA;
obtaining a second 2D NMR spectrum of the second preparation;
comparing one or more cross-peaks in the first 2D NMR spectrum with one or more corresponding cross-peaks in the second 2D NMR spectrum to produce a representation; and
processing the preparation of the test glycoprotein drug substance as drug product if the representation is tolerable; or
taking alternative action if the representation is not tolerable.

49. The method of claim 48, wherein the test glycoprotein is a test antibody and the target glycoprotein is a target antibody.

50. The method of claim 49, wherein the first preparation comprises 10 mg/mL to 150 mg/mL of the test antibody and/or the second preparation comprises 10 mg/mL to 150 mg/mL of the target antibody.

51. The method of claim 48, wherein the step of comparing comprises performing a linear regression analysis.

52. The method of claim 51, wherein the representation is tolerable if the R² value is at least 0.9.

53. The method of claim 48, wherein the first and second NMR spectra are 2D ¹H-¹³C correlation spectra.

54. The method of claim 53, wherein the 2D ¹H-¹³C correlation spectra are ¹H-¹³C heteronuclear multiple quantum coherence (HMQC) spectra.

55. (canceled)

56. The method of claim 48, wherein the processing step comprises one or more of: formulating the test protein, test glycoprotein, or test antibody; processing the test protein, test glycoprotein, or test antibody into a drug product; combining the test protein, test glycoprotein, or test antibody with a second component, e.g., an excipient or buffer; changing the concentration of the test protein, test glycoprotein, or test antibody in the preparation; lyophilizing the test protein, test glycoprotein, or test antibody; combining a first and second aliquot of the test protein, test glycoprotein, or test antibody to provide a third, larger, aliquot; combining the batch of the test protein, test glycoprotein, or test antibody with one or more additional batches of the test protein, test glycoprotein, or test antibody; dividing the test protein, test glycoprotein, or test antibody into smaller aliquots; disposing the test protein, test glycoprotein, or test antibody into a container, e.g., a gas or liquid tight container; packaging the test protein, test glycoprotein, or test antibody; associating a container comprising the test protein, test glycoprotein, or test antibody with a label (e.g., labeling); shipping or moving the test protein, test glycoprotein, or test antibody to a different location.
